(19) United States
(12) Patent Application Publication
(10) Pub. No.: US 2022/0397569 A1
(43) Pub. Date: Dec. 15, 2022

(54) ASSAY FOR RAPID EVALUATION OF CHOROIDAL MAST CELL DEGRANULATION

(71) Applicant: THE JOHNS HOPKINS UNIVERSITY, Baltimore, MD (US)

(72) Inventors: Gerard Lutty, Hyattsville, MD (US); Shuntaro Ogura, Baltimore, MD (US)

(21) Appl. No.: 17/769,960

(22) PCT Filed: Oct. 16, 2020
(86) PCT No.: PCT/US20/56023

§ 371 Date: Apr. 18, 2022

Related U.S. Application Data
(60) Provisional application No. 62/916,981, filed on Oct. 18, 2019.

(51) Int. Cl. G01N 33/50; G01N 33/563

(52) U.S. Cl. CPC G01N 33/5047; G01N 33/563; G01N 2333/4727; G01N 2333/918; G01N 2800/164

(57) ABSTRACT

The present invention relates to the field of ophthalmology. More specifically, the present invention provides compositions and methods useful for screening for drugs to treat age-related macular degeneration (AMD) including geographic atrophy (GA). In one embodiment, a method comprises the steps of (a) administering a drug to a mammal, wherein the mammal comprises a rat or a mouse; (b) enucleating the eyes of the mammal; (c) removing the anterior eye and excising the retina from the eye, wherein the eye comprises an eyecup that comprises choroidal mast cells (MCs); and (d) measuring mast cell degranulation. In an alternative embodiment, a method of the present invention can comprise the steps of (a) contacting an eyecup of a mammal with a drug, wherein the eyecup comprises choroidal mast cells; and (b) measuring MC degranulation.

### CROSS-REFERENCE TO RELATED APPLICATIONS

This application claims the benefit of U.S. Provisional Application No. 62/916,981, filed Oct. 18, 2019, which is incorporated herein by reference in its entirety.

### STATEMENT OF GOVERNMENTAL INTEREST

This invention was made with government support under grant no. EY016151, awarded by the National Institutes of Health. The government has certain rights in the invention.

### FIELD OF THE INVENTION

The present invention relates to the field of ophthalmology. More specifically, the present invention provides compositions and methods useful for screening for drugs to treat age-related macular degeneration (AMD) including geographic atrophy (GA).

### BACKGROUND OF THE INVENTION

Mast cells (MCs) are key effector cells of innate immunity and are resident inflammatory cells in many tissues.¹ Mature MCs express a tyrosine kinase receptor c-KIT and high-affinity IgE receptors on their cell membrane through which MC degranulation is triggered in IgE-mediated hypersensitivity reactions.² Degranulation of MCs is known to release histamine, cytokines, chemokines, and proteases.³ These proteases include MC-specific tryptase and chymase, which initiate an immediate hypersensitivity response to allergens and can activate matrix metalloproteinases (MMPs) that degrade stroma and basement membranes.⁴ Historically MCs have been implicated in asthma,⁵ and recent studies have revealed the significance of MC degranulation in the experimental cardiovascular disease models such as deep vein thrombosis, abdominal aortic aneurysms, and atherosclerosis.⁶⁻⁸ In the eye, MCs are abundant in the conjunctiva and uveal tract, which includes the choroid, but are absent in the retina.^{1,9} Choroidal MCs are analogous to connective tissue MCs in the skin and mesentery in many ways. They respond to compound 48/80, a snake venom-like compound^{1,9} that induces MC degranulation. Their abundance and location in mammalian choroids suggest that MCs can exert local control of choroidal immunologic and inflammatory reactions as well as basic physiological function,¹ however, little is known about the significance of MC degranulation in the choroid.

Age-related macular degeneration (AMD) affects around 200 million people worldwide and is the leading cause of irreversible blindness and visual impairment in developed countries.¹⁰ Geographic atrophy (GA) is the advanced dry form of AMD and is characterized by retinal pigment epithelium (RPE) loss and choriocapillaris attenuation, photoreceptor death, and choroidal thinning. While GA is responsible for 20% of the legal blindness in North America,¹¹ to date, there are no proven treatments and experimental animal models for GA are very limited.¹² Therefore, new therapies and reliable animal models to evaluate the drug efficacy for GA are urgently needed.

### SUMMARY OF THE INVENTION

The present invention is based, at least in part, on the development of an assay for rapid assessment of drugs that may stabilize choroidal mast cells and reduce MC degranulation. Briefly, eyes were enucleated from Sprague-Dawley rats and the anterior eye removed and retina excised. The remaining eyecup was split in half along the central meridian to minimize the use of rats. The half eye cups were incubated at 37° C. in minimal essential medium in the wells of 48 well plates. MCs were stimulated with either (1) compound 48/80 (a snake venom-like compound) or (2) calcium ionophore (CI), and are co-treated with MC stabilizer drugs. The eyecups are incubated for about 180 minutes before processing. Choroidal MCs were stained with non-specific esterase and the percentage of MC DG was evaluated ex vivo. Macrophages (MPs) were co-stained with an anti-Iba1 antibody and their cell volume and sphericity were measured, indices of macrophage activation. Significance of the drug effect was determined with ANOVA analysis.

The present inventors found that MC DG occurs rapidly (as soon as 15 min). By 90 min, the macrophages in choroid are mostly activated. If a drug stabilizes MCs, MCs are mostly non-degranulated and macrophages are not activated after 3 hours incubation. The effect can be quantified by counting the percentage of MCs degranulated and the sphericity and volume of the macrophages. Activated macrophages are smaller and more round than quiescent macrophages (McLeod et al, 2016).

The present invention provides drug evaluation in 180 min as opposed to 6 and 10 weeks, which is required with the present inventors' in vivo rat model readouts. This approach also reduces the number of rats required to test a drug. The present invention allows the effect of drug on MC DG as well as macrophage activation to be assessed rapidly. In further embodiments, the route of administration for a drug can also be tested rapidly. For example, animals can receive drugs for 5 days via different routes of administration and then the effectiveness of the route of administration and the drug can be evaluated by using the eye cups from the dosed animals in the assay.

As described herein, MC quiescence by drugs prevented activation of macrophages (increased sphericity, decreased volume), indicating that MC cytokine release was prevented and the stimuli act only on MCs not MPs. The ex vivo model system of the present invention provides new opportunities for rapid screening of drugs targeting MCs and subsequent ocular inflammation, which MCs stimulate. The present inventors anticipate that this assay will be used to screen drugs for treatment of AMD in which an increase in MC degranulation in human choroid and an increase in macrophage activation has been observed, even prior to loss of RPE cells and thinning of choroid, two characteristics of dry AMD or geographic atrophy.

### BRIEF DESCRIPTION OF THE FIGURES

FIG. 1A-1E. Choroidal MC degranulation in human GA and in the rat model of choroidal MC degranulation. FIG. 1A: Choroidal flat mount immunohistochemistry showing choriocapillaris (UEA lectin; green) and MCs (anti-tryptase; red) in a human GA subject in the non-atrophic region, at the border of atrophy, and in the atrophic lesion. FIG. 1B: Quantification of percentage MC degranulation, as assessed by NSE staining, in the border region of the human GA subjects compared to aged control (n=4, per group). FIG. 1C: The percentage of degranulated MCs over time after subconjunctival implantation of the 48/80-hydrogel in the rat on the implanted or gel region (superior choroid) and the non-gel region (inferior choroid), as assessed with Alcian blue staining. Quantification was performed on the 48/80 eyes throughout the time course and was compared to blank hydrogel eyes (without 48/80) at day 36 (blank, n=3; 48/80, n=4, per time point). FIG. 1D: Time course of MC degranulation in choroidal flat mounts stained with Alcian blue. FIG. 1E: Immunofluorescence of rat choroidal cross sections showing MC (tryptase+; red) and choroidal vessels (GS lectin+; green). Release of tryptase in the choroidal stroma was seen at 14 days post implantation. Data are mean±SD. *P<0.05, **P<0.01, ***P<0.001, 2-tailed, unpaired Student's t test. Scale bars, 20 μm.

FIG. 2A-2F. Morphological changes seen after MC degranulation in the 48/80-hydrogel implanted rats. FIG. 2A: Choroidal flat mount labeled for RPE65. Healthy hexagonal RPE cells decreased with time, area of degenerated RPE expanded, and the presence of enlarged cells with multi-nuclei increased with time. Scale bar, 100 μm. FIG. 2B: Percentage of RPE degenerated in the 48/80-hydrogel implanted eyes on the gel and the non-gel side with time after implantation of 48/80-hydrogel. The blank hydrogel implanted eye values were at 6 weeks (n=12-13, per group). FIG. 2C: Representative cross sections of eyes embedded in glycol methacrylate that received a blank hydrogel or 48/80 on the non-gel, at the border of atrophy, and atrophic region on the gel side at 10 weeks post implantation. Brackets (U) designate outer nuclear layer or photoreceptor nuclei. Scale bar, 50 μm. FIG. 2D: Area of retina and choroid evaluated in cross sections stained with picrosirius red (n=6, per time point). FIG. 2E, 2F: Representative scotopic ERGs at 10 weeks. Graphs showing comparison of amplitude of a- and b-waves with the stimuli of 0.01, 0.1, and 1 cd·s/m2 at 6 weeks (n=9), 8 weeks (n=6) and 10 weeks (n=7) compared to the blank gel (n=8). Data are mean±SD. *P<0.05, **P<0.01, ***P<0.001, 2-tailed, unpaired Student's t test.

FIG. 3A-3F. Morphological analyses after 48/80 implantation in MC deficient rats and WT littermates. FIG. 3A, 3B: Choroidal whole mounts labeled for RPE65 at 6 weeks post implantation of 48/80-hydrogel in MC deficient WsRC^{ws/ws} (^{WS/WS}) and their WT littermate WsRC^{+/+} (^{+/+}) rats. The graph shows quantification of percentage RPE degeneration (n=4, per group). Scale bar, 100 μm. FIG. 3C: Images of three-dimensional volume renderings of macrophage/monocytes (Iba1+; green) and MCs (tryptase+; red) in the choroid. No MCs were detected in WsRC^{ws/ws}, while MCs were degranulated in WsRC^{+/+} WT rats at 6 weeks. FIG. 3D: The graph shows comparison of volume and sphericity of Iba+ cells in WsRC^{+/+} (n=327) and WsRC^{ws/ws} (n=162). Scale bar, 50 μm. FIG. 3E, 3F: Representative scotopic electroretinogram (ERG) with the stimuli of 0.01, 0.1 and 1 cd·s/m2 at 6 weeks. The graph shows b-wave amplitude with the stimulus of 0.01, 0.1 and 1 cd·s/m2 at baseline (n=4, per group) and 6 weeks post implantation (n=8, per group). Data are mean±SD. *P<0.05, **P<0.01, ***P<0.001, 2-tailed, unpaired Student's t test.

FIG. 4A-4G. Assays to evaluate MC degranulation and drug efficacy. FIG. 4A: In vitro microplate assay evaluating 48/80-induced (10 μg/mL) release of β-hexosaminidase from RBL-2H3 cells. Cells were co-incubated with 48/80 with or without ketotifen fumarate (Kef) (10 μg/mL) or untreated (no 48/80) medium for 45 minutes (n=4). FIG. 4B, 4C: Choroidal flat mount from ex vivo eyecup assay in MC deficient WsRC^{ws/ws} (^{WS/WS}) rats showing macrophages (Iba1+) and MC (tryptase+). Eyecups were incubated for 3 hours with 48/80 (300 μg/mL). MCs were not detected in these rats. Graph showing comparison of volume and sphericity of Iba1+ cells in untreated and 48/80 treated eyes of WsRC^{ws/ws} rats (untreated, n=165; 48/80 treated, n=286). FIG. 4D: Bright field image of the choroid showing MCs (NSE+) after 3 hours ex vivo in S/D rats. The boxes are higher magnification images of MCs. FIG. 4E: Quantification of 48/80-induced MC degranulation after 3 hours, co-incubated with or without ketotifen fumarate and untreated (n=4, per group). FIG. 4F: Ex vivo immunohistochemistry of S/D choroid showing MC [NSE, auto-fluorescence (AF)] at 633 nm wavelength and macrophages (Iba1+) after 3 hours treatment. FIG. 4G: Comparison of cell volume and sphericity of Iba1+ cells treated with 48/80 with or without ketotifen fumarate and untreated (untreated, n=114; 48/80, n=127; 48/80 with ketotifen fumarate, n=112). Data are mean±SD. ***P<0.001, one-way ANOVA with Tukey post hoc comparison (FIG. 4A, 4E, 4G), 2-tailed, unpaired Student's t test (4C). Scale bars, 20 μm

FIG. 5A-5I. Results of quiescing MCs with the generic MC stabilizer, ketotifen fumarate, in the rat model. FIG. 5A, 5B: Choroidal whole mount labeled for RPE65 at 4 weeks post implantation of 48/80-hydrogel in rats treated orally twice daily with ketotifen fumarate (Kef) or PBS. The graph shows quantification of percentage RPE degeneration in S/D rats (n=6, per group). FIG. 5C-5E: Bright field images of the choroid which show MCs (NSE+) (top) and the images of three-dimensional volume renderings shows choroidal macrophage (Iba1+) (bottom) in ketotifen fumarate and PBS treated rats 4 weeks after implanting 48/80-hydrogel. The graph shows percentage of MC degranulation in the two groups (n=6, per group) and comparison of the volume and sphericity of Iba1+ cells in the choroid after treating with ketotifen fumarate and PBS for 4 weeks (ketotifen fumarate, n=570; PBS, n=601). FIG. 5F, 5G: Representative ERG with the stimuli of 0.01, 0.1 and 1 cd·s/m2 at 8 weeks after treating with ketotifen fumarate or PBS in the 48/80 implanted eyes. The graph shows a- and b-wave amplitude blank-hydrogel implanted eyes and ketotifen fumarate or PBS treated eyes with the stimulus of 0.01, 0.1, and 1 cd·s/m2 at 8 weeks post implantation (n=7, per group). FIG. 5H, 5I: Representative H&E sections of blank-hydrogel implanted eye and treatments with ketotifen fumarate and PBS for 8 weeks in the rat model. The graphs show area of retina and choroid evaluated in cross sections stained with picrosirius red (n=6, per group). Data are mean±SD. *P<0.05, **P<0.01, ***P<0.001, 2-tailed, unpaired Student's t test. Scale bars, 50 μm

FIG. 6A-6H. Effects of inhibiting MC-derived tryptase in the rat model. FIG. 6A, 6B: ARPE-19 cells labeled with phalloidin after 72 hours treatment with serum free media (control), tryptase 1 and 5 μg/mL in vitro. The graph shows quantification of the percentage of ARPE-19 cells that have degenerated (n=4, per group). FIG. 6C, 6: Choroidal whole mounts labeled for RPE65 at 4 weeks post implantation of 48/80-hydrogel in rats treated with daily subcutaneous injections of the tryptase inhibitor APC 366 or vehicle. The graph shows quantification of the percentage of RPE that have degenerated (APC 366; n=8, vehicle; n=6). FIG. 6E, 6F: ERG with the stimulus of 0.01, 0.1 and 1 cd·s/m2 at 8 weeks after treating with APC 366 or vehicle in the 48/80 implanted eyes. The graph shows a- and b-wave amplitude with the stimuli of 0.01, 0.1 and 1 cd·s/m2 at 8 weeks post implantation treated with APC 366 or vehicle (n=8, per group). FIG. 6G, 6H: Representative H&E sections after treating with APC 366 and vehicle for 8 weeks after administrating 48/80 in the rat. The graphs show area of retina and choroid evaluated in cross sections stained with picrosirius red (n=6, per group). Data are mean±SD. *P<0.05, **P<0.01, ***P<0.001; one-way ANOVA with Tukey post hoc comparison (FIG. 6B), 2-tailed, unpaired Student's t test (FIG. 6D, 6F, 6H). Scale bars, 50 μm

FIG. 7. Schematic of hypothesis that MCs contribute to GA. The working hypothesis is that choroidal MCs are activated by hypoxia-induced IL-33 production by RPE or CRP, AGEs, C3a or C5a, which are known to be elevated in AMD choroid. Upon activation and degranulation, tryptase is released into choroidal stroma and Bruch's membrane. Tryptase can degrade choroidal stroma and Bruch's membrane resulting in thinning of choroid and degeneration of RPE, which are hallmarks of GA

FIG. 8A-8B. Implantation of compound 48/80-hydrogel. FIG. 8A: In vitro release kinetics of compound 48/80 from hydrogel in PBS at 37° C. as measured with the BCA assay. Within 48 h, ˜98% of the 48/80 was released in aqueous conditions. FIG. 8B: Choroidal flat mount with MCs stained for NSE enzyme activity. The hydrogel was implanted in the superior subconjunctiva. MCs were distributed along the long posterior ciliary arteries longitudinally through the choroid. Scale bar, 1 mm.

FIG. 9A-9C. RPE cell changes after MC degranulation. FIG. 9A: Choroidal flat mount immunofluorescence showing RPE65 and bright field images of the same choroidal area showing MC (NSE⁺) in the non-gel and gel side 2 wk after 48/80 implantation. Degenerated RPE cells were observed where MCs had degranulated. FIG. 9B: Choroidal flat mounts stained for RPE65 (red) and autofluorescence of lipofuscin detected at the 488 nm wavelength (green) 4 wk post implantation. FIG. 9C: Choroidal flat mounts labeled for RPE65 and ZO-1 4 wk after implantation of 48/80. RPE degeneration was apparent because junctions were disturbed and healthy hexagonal shape of RPE cells was disrupted in 48/80 implanted choroid. Scale bars, 20 μm.

FIG. 10A-B. Phenotypes of MC deficient rats. FIG. 10A: Conjunctiva stained with NSE in MC deficient WsRC^{WS/WS} (^{WS/WS}) and their WT littermate WsRC^{+/+} (^{+/+}) rats. There was no MC staining in the MC deficient WsRC^{WS/WS} rats. FIG. 10B: Immunohistochemistry of choroid showing RPE65 and macrophage/microglia (Iba1⁺) in WsRC^{WS/WS} and WsRC^{+/+} rats at 6 wk post 48/80 implantation (top) and orthogonal images (bottom). Supra-RPE or subretinal macrophage/microglia were detected in WsRC^{+/+} rats in areas with RPE degeneration, which is often seen in human GA. Scale bars, 20 μm.

FIG. 11A-11D. Ex vivo assays to evaluate MC and macrophages. FIG. 11A: Choroidal flat mounts of ex vivo eye cups in WT WsRC^{+/+} (^{+/+}) rats, the controls for the MC deficient rats, showing macrophages (Iba1⁺) and MCs (tryptase⁺). Eyecups were incubated for 3 h with 48/80 (300 μg/ml). FIG. 11B: Comparison of volume and sphericity of Iba1⁺ cells in WsRC^{+/+} rats in untreated and 48/80 treated eye cups (untreated, n=130; 48/80 treated, n=72). See also FIG. 4B, 4C. FIG. 11C: Time course of changes in MCs and macrophages in ex vivo eye cup assay after treating with 48/80 (300 μg/ml). Bright field images of the choroid show MC (NSE⁺) while three-dimensional volume renderings show shape changes in choroidal macrophages (Iba1⁺). FIG. 11D: Time course of cell volume and sphericity changes in Iba1⁺ cells after 48/80 treatments (untreated, n=131; 10 min, n=255; 30 min, n=272; 60 min, n=285; 90 min, n=176). Data are mean±SD. *P<0.05, **P<0.01, ***P<0.001, 2-tailed, unpaired Student's t test (FIG. 11B), one-way ANOVA with Tukey post-hoc comparison (FIG. 11D). Scale bars, 50 μm.

FIG. 12. Ex vivo assay after treating with ketotifen fumarate. Ex vivo eyecups from rats treated with either ketotifen fumarate (15 mg/kg) or PBS, twice daily for 4 days. Eyecups were untreated (no 48/80, i.e., just medium) or treated with 48/80 (300 μg/ml) on the fifth day. Bright field images of the choroid show MCs (NSE⁺) and the images of three-dimensional volume renderings shows choroidal macrophage (Iba1⁺). The graphs show quantification of percentage of MCs degranulated (Untreated+Kef, n=3; 48/80+Kef, n=3; 48/80+Kef, n=5) and volume and sphericity of Iba1⁺ cells after treatments (untreated+Kef, n=305; 48/80+PBS, n=372; 48/80+Kef, n=387). Data are mean±SD. **P<0.01, one-way ANOVA with Tukey post-hoc comparison. Scale bars, 50 μm.

FIG. 13A-13C. Effects on rats treated with daily ketotifen fumarate. FIG. 12A: Immunofluorescence in a representative choroidal flat mount labeled for RPE65 after twice daily treatment with oral ketotifen fumarate (15 mg/kg) to normal S/D rats for 6 wk. No apparent RPE degeneration was detected. FIG. 12B: Comparison of the body weights of rats treated with ketotifen fumarate or PBS twice daily for 6 wk after 48/80 implantations. (n=7, per group). FIG. 12C: Amplitude of a- and b-waves in ERG with the stimulus of 0.01, 0.1, and 1 cd·s/m² of normal (non-48/80 injected) rats treated with ketotifen fumarate for 6 wk and age matched untreated rats (n=4, per group). Data are mean±SD. 2-tailed, unpaired Student's t test. Scale bar, 100 μm.

FIG. 14A-14B. Prevention of macrophage activation after APC 366 injections. FIG. 14A: Representative images of three-dimensional volume renderings showing choroidal macrophage (Iba1⁺) after 4 wk daily treatment with either MC-tryptase inhibitor APC 366 or vehicle control in 48/80 implanted eyes. FIG. 14B: Graphs show comparison of the volume and sphericity of Iba1⁺ cells after treatments (APC 366, n=696; vehicle, n=802). Data are mean±SD. ***P<0.001, 2-tailed, unpaired Student's t test. Scale bar, 20 μm.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

Recently, the present inventors observed an increase in number and percentage of degranulated choroidal MCs in all types of AMD subjects.¹³ The possible reason for this is the proinflammatory milieu of AMD choroid, which includes elevated complement factor C3a, C5a, C-reactive protein (CRP), and advanced glycation end products (AGEs), all of which can stimulate MC degranulation.¹⁴⁻¹⁶ Human choroidal MCs release tryptase and histamine when they degranulate,¹⁷ and tryptase is released specifically from MCs in the first wave of granules during degranulation.¹⁸ Tryptase degrades collagens and activates MMPs, which degrade stroma and basement membranes. In GA, MC-derived tryptase was localized to Bruch's membrane, vascular basement membranes and throughout the choroidal stroma near degranulated MCs.¹⁷ These observations imply MC involvement in GA etiology, however, there is still a void in the understanding of how MC degranulation contributes to the progression of GA.

The purpose of the current study was to determine if chronic choroidal MC degranulation results in changes that were phenotypic characteristics of GA. Therefore, RPE atrophy as well as reduced retinal function, and retinal and choroidal thinning were assessed after inducing MC degranulation in a rat model. To evaluate if MCs could be a therapeutic target in GA, ketotifen fumarate, a generic MC stabilizer, was evaluated for prevention of MC degranulation in in vitro and ex vivo assays for MC degranulation, and in the present inventors' in vivo long-term rat model of GA. In addition, a MC tryptase inhibitor was also evaluated in the rat model. The present inventors' results suggest for the first time, choroidal MC involvement in the development of GA and MCs as a potential therapeutic target for GA.

Accordingly, in one aspect, the present invention provides compositions and methods useful for screening drugs that stabilize MCs and thus, prevent degranulation. In one embodiment, a method comprises the steps of (a) administering a drug to a mammal, wherein the mammal comprises a rat or a mouse; (b) enucleating the eyes of the mammal; (c) removing the anterior eye and excising the retina from the eye, wherein the eye comprises an eyecup that comprises choroidal mast cells (MCs); and (d) measuring mast cell degranulation.

In another embodiment, administering step (a) further comprises administering an agent that promotes MC degranulation. In a specific embodiment, the agent comprises compound 48/80.

In a specific embodiment, step (d) comprises staining for non-specific esterase (NSE) activity in mast cells present in the choroid of the eyecup. In a more specific embodiment, the method further comprises counting non-degranulated and granulated cells in the whole choroid, wherein degranulated cells exhibit irregular shape or extracellular granules

In another embodiment, the method further comprises staining macrophages and measuring macrophage cell volume and sphericity. In a specific embodiment, macrophages are stained with anti-Iba1 antibody.

In yet another embodiment, the method further comprises staining for tryptase with an anti-MC tryptase antibody.

In particular embodiments, the drug is administered to the mammal at least twice over the course of 1-7 days.

A method of the present invention can comprise the steps of (a) contacting an eyecup of a mammal with a drug, wherein the eyecup comprises choroidal mast cells; and (b) measuring MC degranulation. In particular embodiments, the eyecup has been separated from the anterior eye and retina.

In another embodiment, contacting step (a) further comprises contacting the eyecup with an agent that promotes MC degranulation. In a specific embodiment, the agent comprises compound 48/80.

In a specific embodiment, step (b) comprises staining for NSE activity in mast cells present in the choroid of the eyecup. In a more specific embodiment, the method further comprises counting non-degranulated and granulated cells in the whole choroid, wherein degranulated cells exhibit irregular shape or extracellular granules.

In another embodiment, the method further comprises staining macrophages and measuring macrophage cell volume and sphericity. In a specific embodiment, macrophages are stained with anti-Iba1 antibody.

In another embodiment, the method further comprises staining for tryptase with an anti-MC tryptase antibody.

In particular embodiments, the time between steps (a) and (b) comprises at least 90 minutes. In a more specific embodiment, the time between steps (a) and (b) comprises about 180 minutes.

The methods of the present invention can include administering mast cell stabilizers as a control. Examples of such compounds include, but are not limited to, β2-adrenergic agonists, cromoglicic acid, ketotifen, methylxanthines, olopatadine, rupatidine, mepolizumab, omalizumab, pemirolast, quercetin, nedocromil, azelastine, tranilast, palmitoylethanolamide, and vitamin D.

Without further elaboration, it is believed that one skilled in the art, using the preceding description, can utilize the present invention to the fullest extent. The following examples are illustrative only, and not limiting of the remainder of the disclosure in any way whatsoever.

### Examples

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

The experiments described below sought to determine if mast cells (MCs) contribute to geographic atrophy (GA), an advanced form of age-related macular degeneration. The cause of GA is elusive and there is currently no therapy for this blinding disorder. Mast cells (MCs) are the initial responders of innate immunity and are involved in many diseases. This study demonstrates that choroidal MC degranulation and MC-derived tryptase could be involved in both human GA and a rat model for GA. Continuous activation of MCs in a slow release fashion in the rat induced characteristic phenotypes of human GA: retinal pigment epithelium degeneration followed by retinal and choroidal thinning. Pharmacological intervention targeting MC degranulation with ketotifen fumarate or inhibition of MC-derived tryptase with APC 366 reduced MC degranulation and prevented all of GA-like phenotypes in the rat model. These results suggest choroidal MC involvement in GA etiology and MCs as a potential target for GA.

### Materials and Methods

Human eyes. Human donor eyes were obtained from the National Disease Research Interchange (Philadelphia, Pa., USA). All tissues were obtained within 10-35 hours of death. All donors were Caucasian. GA was diagnosed when a distinct area of RPE degeneration with sharply defined borders without apparent neovascularization. Utilization of the human tissue was in accordance with the Declaration of Helsinki with approval of the Joint Committee on Clinical Investigation at Johns Hopkins University School of Medicine.

Rats. Sprague/Dawley male rats (200-250 g) were purchased from Envigo (Frederick, Md., USA). MC deficient WsRC^{WS/WS} rats and their littermate wild-type (WT) WsRC^{+/+} rats were purchased from Japan SLC (Hamamatsu, Japan). For all procedures, anesthesia was performed by intramuscular injection of a ketamine (100 mg/mL) and xylazine (100 mg/mL) cocktail. Pupils were dilated with 1% tropicamide and 2.5% phenylephrine eye drops followed by topical anesthesia. All animal experimental procedures were performed according to the Association for Research in Vision and Ophthalmology Statement for the Use of Animals in Ophthalmic and Vision Research, following approval from the Animal Care and Use Committee at the Johns Hopkins University.

Images. Images were captured using Zeiss LSM710 confocal microscope with ZEN software (Carl Zeiss, Jena, Germany), Zeiss Photomic II microscope equipped with QCapture Imaging camera and software (Teledyne QImaging, Surrey, BC, Canada), or Leica AF6000 with Leica Application Suite software (Leica Microsystems, Buffalo Grove, Ill., USA). Images were analyzed using NIH ImageJ (version 1.50), Adobe Photoshop (CS4 and CS6, Adobe Systems, San Jose, Calif.), and Imaris (version 8.3.1, Bitplane USA, Concord, Mass., USA).

Human flat mount preparation. MCs in human choroidal flat mounts were stained for nonspecific esterase (NSE) and alkaline phosphatase (APase) enzyme activities as previously described.¹³ Briefly, after the retina was carefully removed, eyecup with choroid was soaked in 1% EDTA (Thermo Fisher Scientific, Waltham, Mass., USA) in distilled water for 2 hours at room temperature (RT) to remove RPE. Any adherent RPE cells were removed by squirting EDTA solution from a syringe with a blunted 25-gauge needle. RPE-denuded choroids were then isolated from the sclera, and fixed for 1 hour in 2% paraformaldehyde (PFA) in 0.1 M cacodylate buffer at RT and incubated for the APase activity as previously described.¹³ After APase staining, NSE staining was performed using a naphthol AS-D chloroacetate kit (91C-1KT, Millipore Sigma, St Louis, Mo., USA).¹³ Granulocytes and MC stained red with this method, but the two cell types were easily distinguished due to their size difference. Then, APase and NSE double stained choroids were postfixed in 2% PFA for 24 hours, washed, and exposed to 30% hydrogen peroxide (Millipore Sigma, St Louis, Mo.) to bleach the melanin at 4° C. as previously reported.13 Total MC number and degranulated MCs at the border of GA and aged matched eyes were counted in five 1 mm² areas and averaged per eye for quantification.

For flat mount IHC choroids, eyes were fixed in 2% PFA in TBS overnight after RPE removal.17 After several washes, choroids were incubated with 5% normal goat serum in 0.1% triton X-100 in TBS with 1% BSA overnight at 4° C., followed by incubation with mouse anti-MC tryptase antibody (Ab)(1:500, ab2378, Abcam, Cambridge, Mass., USA) for 72 hours at 4° C. After washes, the choroid was incubated for 48 hours at 4° C. with goat anti-mouse Ab conjugated with Cy3 (1:200, Jackson ImmunoResearch, West Grove, Pa., USA) and Ulex europaeus agglutinin I (UEA) lectin conjugated with FITC (1:100, GTX01512, GeneTex, Irvine, Calif., USA) and then, imaged with a Zeiss LSM710.

Rat flat mount preparation. After enucleating the eyes, the anterior segments were removed and the retina was carefully excised. Eyecups were then fixed overnight with 2% PFA at 4° C. After washing, four pie cuts were made to allow flattening of the choroid/sclera eyecup as well as clearly isolate superior and inferior quadrants. Then the choroid was prepared for IHC as previously described.¹⁹ The choroid was blocked with 2% normal goat or donkey serum for 4 hours at 4° C., washed in 0.1% triton X-100 in TBS, and then, incubated with a mixture of the following primary Abs: mouse anti-RPE65 (1:200, NB100-355, Novus Biologicals, Centennial, Colo., USA), goat anti-Iba1 (1:200, ab5076, Abcam, Cambridge, Mass., USA), and rabbit anti-MC tryptase (1:200, CAU26568, Biomatik, Wilmington, Del., USA) overnight at 4° C. After washing, they were incubated with Alexa Fluor 488 or Cy3 conjugated goat or donkey secondary Abs (1:300, Jackson ImmunoResearch, West Grove, Pa., USA) overnight, followed by mouse anti-ZO-1 Ab conjugated with Alexa Fluor 594 (1:100, 339194, Thermo Fisher Scientific, Waltham, Mass., USA).

Rat cross section preparation. Rat eyecups were cryopreserved as reported previously.¹⁷ Eight μm cryosections were permeabilized with absolute methanol at −20° C. and blocked with 2% normal donkey serum at RT. After washing, sections were incubated for 2 hours at RT with rabbit anti-MC tryptase Ab (1:200, CAU26568, Biomatik, Wilmington, Del., USA). After washing, sections were incubated for 30 minutes at RT with Isolectin GS-IB4 from Griffonia simplicifolia (GS lectin) conjugated with Alexa Fluor 488 (1:100, 121411, Thermo Fisher Scientific, Waltham, Mass., USA) and donkey anti-rabbit Ab conjugated with Alexa Fluor 647 (1:500, Jackson ImmunoResearch, West Grove, Pa., USA) and then, coverslipped with mounting medium (Vector Laboratories, Burlingame, Calif., USA).

Hydrogel preparation and injection. A hydrogel which slowly released the compound 48/80 (Millipore Sigma, St Louis, Mo., USA), a snake venomlike compound, was formulated using thiolated hyaluronic acid and 4-arm poly (ethylene glycol) acrylate crosslinked using thiol-ene click chemistry.20 The gel consisted of 48/80 (20 mg/mL), 4-arm PEG-acrylate (100 mg/mL, PSB-421, Creative PEGWorks, Chapel Hill, N.C., USA), 8-arm PEG SH (150 mg/mL, PSB-851, Creative PEGWorks, Chapel Hill, N.C.), hyaluronic acid-SH (200 mg/mL, HA-371, Creative PEGWorks, Chapel Hill, N.C.) added to Irgacure 2959 (Ciba-Geigy, Tarrytown, N.Y., USA). All components were then mixed together by vortex and placed on ice. The final solution was loaded into an insulin syringe with a 31-gauge needle (BD Biosciences, San Jose, Calif., USA) and then, exposed to UV light (AnalytikJena, Upland, Calif., USA) for 1.5 minutes. The release and stability of the hydrogel were evaluated in vitro in PBS, pH 7.4 at 37° C.20. The amount of 48/80 released was measured using bicinchoninic acid (BCA) assay. After confirming the gel formation by its consistency, 30 μL of hydrogel with or without 48/80 (blank hydrogel) was implanted into the superior subconjunctival space.

Alcian blue staining. After RPE removal, eyecups were fixed in methanol-formalin-acetic acid cocktail for 30 minutes and MCs were further stained with 0.05% Alcian blue (pH 5.0) in 0.02 M sodium acetate buffer for 45 minutes. Choroids were isolated after eyecups were split at the meridian and the number of non-degranulated and degranulated MCs were counted.

RPE degeneration. After the choroid/sclera eyecup was stained with anti-RPE65 Ab, 4-5 fields from the gel and non-gel areas of each choroid were captured as optimized z-stacks at 10 or 20× magnification with Zeiss LSM710. Images were exported to ImageJ software, converted into 8-bit gray color and then, thresholded. The dark area was considered degenerating RPE. To exclude the area of RPE nucleus (unstained with anti-RPE65 Ab, therefore dark) from the total dark area, particle sizes ranging from 100-infinity (pixels²) were analyzed so nuclei were excluded. Percentage of RPE degeneration was averaged per choroid and then, quantified.

Retinal and choroidal thickness. Eight μm cryosections through the optic nerve were stained with Picrosirius Red Stain Kit (Polysciences, Inc, Warrington, Pa., USA) for 5 minutes as recommended by the manufacturer. After rinsing in hydrochloric acid and dehydrating in ethanol, sections were cover-slipped with Permount Mounting Medium (Thermo Fisher Scientific, Waltham, Mass., USA). The sections were then imaged with a Zeiss Photomic II microscope at 10×, and adjacent overlapping images of each section were taken from Ora serrata to Ora serrata. The overlapping images were then stitched using Adobe Photoshop and a panorama image was generated. The entire retina and choroid of each panorama image was precisely hand-traced and areas of the retina and choroid, respectively, were measured using ImageJ.

For morphologic analyses, eyes were cryopreserved and stained with H&E staining as previously described¹⁵ or fixed with 2.5% glutaraldehyde, 2% PFA in 0.1 M cacodylate buffer. After the anterior segments were excised, eyes were cut in half through the optic disk. Then, eyes were washed and dehydrated and embedded in glycol methacrylate and cut with 2 μm thickness as previously published.²¹

Electroretinogram (ERG). After overnight dark adaptation, rats were deeply anesthetized followed by pupil dilation. The rats were placed on a heated platform and full-field scotopic ERGs were elicited by white light flashes with corneal electrodes at intensities ranging from 0.01 to 1 cd·s/m² using the Celeris ERG system (Diagnosys LLC, Lowell, Mass., USA). The amplitude of the a- and b-waves was measured from the a-wave trough to the b-wave peak.

In vitro assays. RBL-2H3 cells (CRL-2256, ATCC, Manassas, Va., USA) were used for the in vitro assay for MC degranulation. Cell degranulation was assessed by the (3-hexosaminidase release as previously described.²² A density of 50 000 cells/well was plated overnight in a 96-well plate. Ten μg/mL of 48/80 or 10 μg/mL of ketotifen fumarate (1(2628, Millipore Sigma, St Louis, Mo., USA)+10 μg/mL of 48/80 dissolved in 100 μL serum free media (DMEM with 1.0 g/L glucose) (Thermo Fisher Scientific, Waltham, Mass.) were added to each well and incubated for 45 minutes. Then, the cells were lysed using 1% triton X-100 to determine MC β-hexosaminidase that was still cell associated. Twenty μL of the supernatant and then, the cell lysate was collected and added to 50 μL of cold 4-Nitrophenyl N-acetyl-B-d-glucosaminide (1.3 mg/mL in Citrate Buffer, pH 4.5, Millipore Sigma, St Louis, Mo., USA) to detect the amount of β-hexosaminidase released. After the plate was incubated for 90 minutes, 100 μL of 0.2 M sodium hydroxide with 0.2 M glycine (pH 10) were added to terminate the reaction. The plate was read at 405 nm wavelength using a microplate reader (ELx808, BioTek, Winooski, Vt., USA). Degranulation was calculated as the percentage of β-hexosaminidase activity measured in the supernatants relative to the total amount of β-hexosaminidase activity in the cells exposed to 1% triton X-100 (100% degranulation).

ARPE-19 cells (CRL-2302, ATCC, Manassas, Va., USA) incubated in a 24-well plate for 14 days were used for assessing the effect of tryptase on confluent RPE cell monolayer. After washing in PBS, cells were treated with 500 μL serum free media, with or without human lung tryptase (650366-M, Millipore Sigma, St Louis, Mo., USA) (1 or 5 μg/mL) for 72 hours. Then cells were fixed with 4% PFA, permeabilized with 0.1% triton X-100 in PBS, and stained with phalloidin-tetramethylrhodamine B isothiocyanate (1:500, Millipore Sigma, St Louis, Mo., USA). Images of four fields in the center of the well were captured as optimized z-stacks at 10× magnification with Zeiss LSM710. Images were exported to ImageJ software, converted into 8-bit gray color and thresholded. Percentage of the ARPE-19 cell monolayer degeneration was averaged per well and quantified.

Ex vivo assays. An ex vivo assay was developed to evaluate drugs quickly and efficiently. After enucleation, the anterior segments and retinas were excised and the eyecups were split at the meridian and divided in halves. Then, eyecups were incubated with 300 μg/mL of 48/80 with or without 300 μg/mL of ketotifen fumarate in serum free DMEM in a 48-well plate for 3 hours. To evaluate oral ketotifen fumarate potential to prevent MC degranulation, rats were treated orally twice daily with ketotifen fumarate (15 mg/kg) in PBS or with PBS as control for 4 days and 2 hours prior to enucleation on the 5th day. Eyes were enucleated and exposed in 48/80 serum free DMEM for 3 hours. After washing, RPE was removed and the eyecup fixed for histochemistry. The choroid was isolated from the sclera and MCs were stained for NSE activity and viewed on the Zeiss LSM710 for the autofluorescence of the reaction product. The numbers of non-degranulated and degranulated cells were counted in the whole choroid. MCs were considered as degranulated when they showed irregular shapes or extracellular granules. For WsRC rats, melanin was bleached using the Melanin Bleaching Kits (Polysciences, Inc, Warrington, Pa.) following the manufacture's protocol before proceeding to immunohistochemistry.

Volumetric and sphericity measurement. Volumetric and sphericity measurements were performed in the rats to analyze changes in shape and size of choroidal macrophages/monocytes stained with Iba1 as previously reported.²³ Briefly, randomly chosen 4-5 fields of optimized 40× magnification z-stacks per choroid captured with Zeiss LSM710 were opened in Surpass View of Imaris software and surfaces (surface detail 1.5 μm) were created, using background subtraction (0.5 μm). Voxel number filtering was applied to remove nonspecific particles in the surfaces created. Cells touching others or ones which had only a portion of the cell body visible were omitted from the volume rendering and then, the cell volume and sphericity were quantified with Imaris software.

Pharmacokinetic study. Ketotifen fumarate quantification in plasma and eye tissue was conducted using high-performance liquid chromatography with tandem mass spectrometry (LC/MS-MS). Briefly, acetonitrile containing 0.5 μM losartan as internal standard was used to extract ketotifen from choroid and retina. Standards were prepared by spiking ketotifen fumarate in naive tissue from 0.003 to 100 μmol/g in a half log dilution series. Samples were weighed and placed in low retention microcentrifuge tubes with 5 μL extraction solution/mg tissue and pestle homogenized. Samples were vortexed followed by centrifugation at 16 000 g for 5 minutes at 4° C. The supernatants were transferred to a 96-well plate and 2 μL were injected for analysis. Samples were analyzed on an UltiMate 3000 UHPLC coupled to Q Exactive Focus orbitrap mass spectrometer (Thermo Fisher Scientific, Waltham, Mass., USA) using an Agilent EclipsePlus C18 RRHD (1.8 μm; 2.1. 100 mm) column. The mobile phase consisted of water+0.1% formic acid, and acetonitrile+0.1% formic acid. Separation was achieved at a flow rate of 0.4 mL/min using a gradient run. Samples were analyzed in positive ion mode via heated electrospray with capillary temperature set at 350° C. and a spray voltage of 3.5 kV. Data acquisition were performed in selected ion monitoring (SIM) mode isolating 310.1260 m/z (ketotifen fumarate) and 423.1695 m/z (losartan) with a 1 m/z isolation window. Data were acquired and quantified with Xcalibur software (Thermo Fisher Scientific, Waltham, Mass., USA).

Drug treatments. Rats were pretreated for 1-2 days prior to 48/80 hydrogel injection with either oral treatment of ketotifen fumarate (15 mg/kg) or PBS twice daily. MC tryptase inhibitor APC 366 (5 mg/kg, CAS 258932-85-8, R&D Systems, Minneapolis, Minn.)²⁴ or control vehicle was administered subcutaneously once daily for 8 weeks.

Statistics. Statistical analyses were performed with Prism 8 (GraphPad Software, San Diego, Calif.) using a 2-tailed Student's t test or one-way ANOVA with a Tukey post hoc comparison test. Data were expressed as mean±SD P values of less than 0.05 were considered statistically significant.

### Results

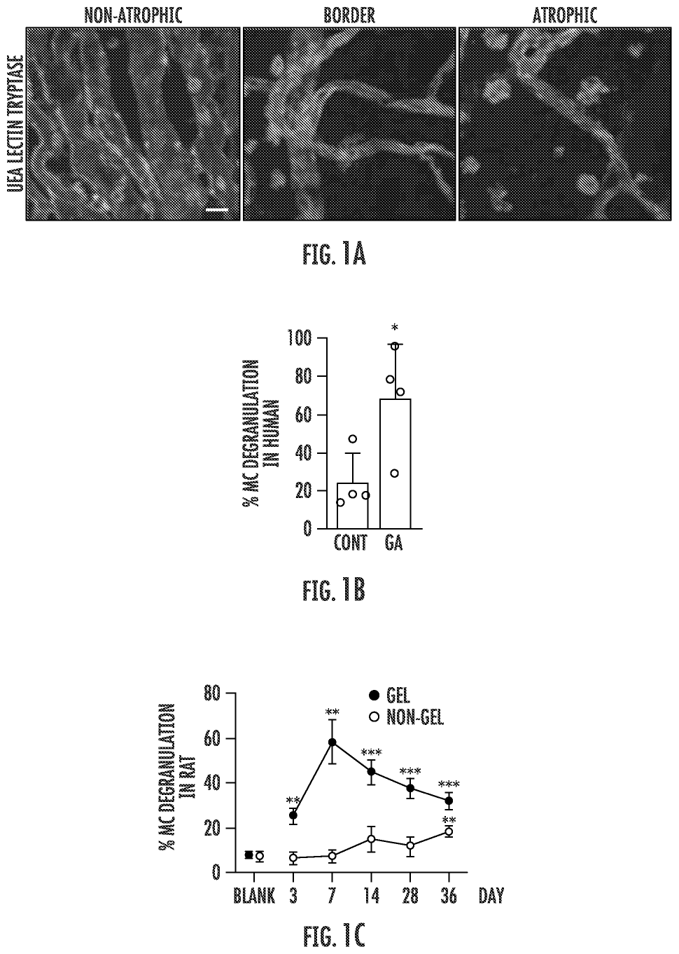

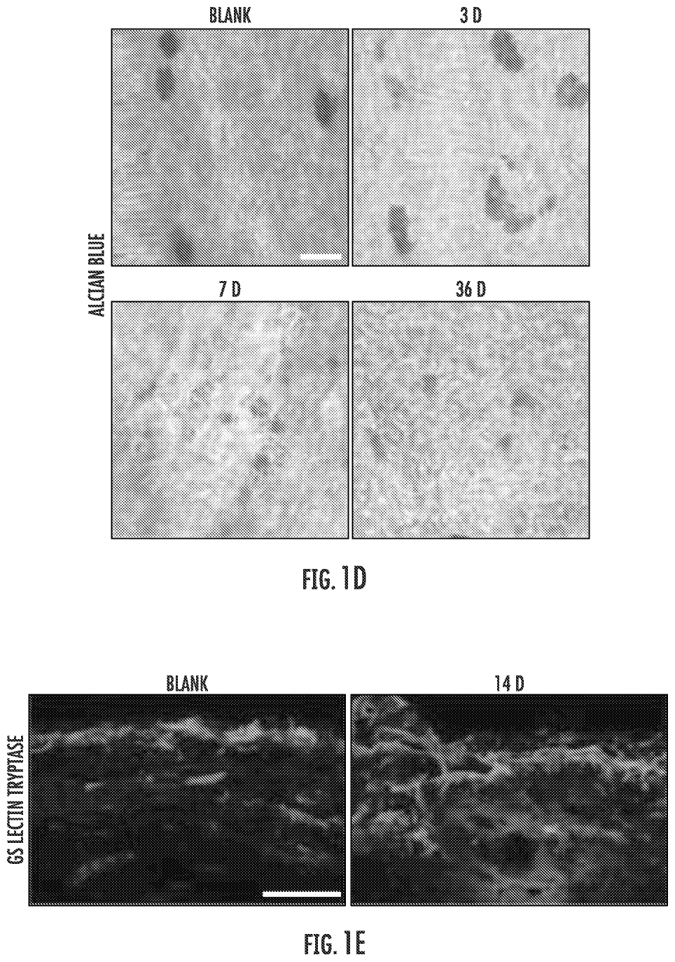

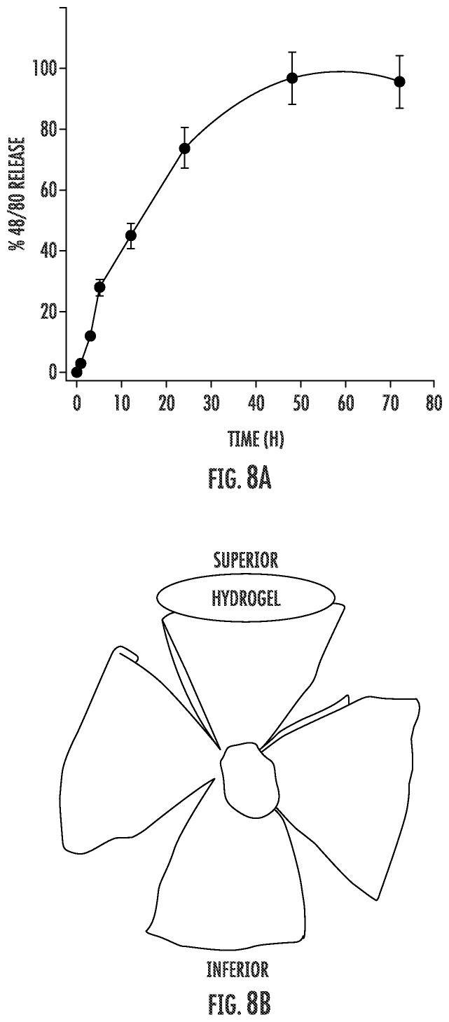

Sustained MC activation by subconjunctival 48/80-hydrogel implantation in the rat eye. Immunohistochemical localization of tryptase in human GA choroids demonstrated that many choroidal MCs were degranulated at the border of non-atrophic and atrophic choroid (FIG. 1A).¹³ In addition, there was a significant increase in percent degranulated MCs at the border of GA, where RPE loss occurs, compared to the age matched healthy eyes (FIG. 1B). Given the results in human subjects, the present inventors next investigated whether MC degranulation could be continuously stimulated in the rat choroid with compound 48/80 (hereafter called 48/80), a snake venom-like compound and a well-established MC stimulator. It was previously reported that direct injection of 48/80 into the subconjunctival space induced anterior segment inflammation within 30 minutes in parallel with MC degranulation, resulting in severe uveitis.²⁵ This suggested that 48/80 must be delivered in slow release fashion from a hydrogel pellet allowing continuous activation and degranulation of choroidal MCs but avoiding acute uveitis. The release and stability of the hydrogel containing 48/80 was first evaluated in vitro utilizing BCA assay. Injectable hydrogels in PBS released 48/80 in a sustained manner for a period of 3 days with an initial burst releasing up to 28% within 5 hours (FIG. 8A). This rapid release was assessed in an aqueous solution but a much slower sustained release was predicted in vivo in the subconjunctival space.

Given the distribution of choroidal MCs primarily along the long posterior ciliary arteries in the rat choroid,^{1,9} the hydrogel with 48/80 was injected subconjunctivally in Sprague/Dawley (S/D) rats in the superior quadrant of the eye, distant from the midline of eye, and the greatest concentration of MCs (FIG. 8B). A blank hydrogel (without 48/80) was injected in control rats. The hydrogel solidified in the subconjunctival space and is called an implant herein. As predicted, a significant MC degranulation in the gel region of 48/80 implanted eyes was observed using Alcian blue stained choroidal flat mounts; the degranulation peaked at 7 days post implantation, and MCs were still degranulated even at day 36 (FIG. 1C,1D). In contrast, no significant degranulation was observed on the non-gel side until day 36, that is, the inferior quadrant distant from the hydrogel, suggesting that hydrogel interacted focally over the 1st week with a gradual diffusion to the inferior choroid (FIG. 1C). Consistent with human GA subjects, tryptase was released from degranulating MCs into the choroidal stroma (FIG. 1E).¹⁷

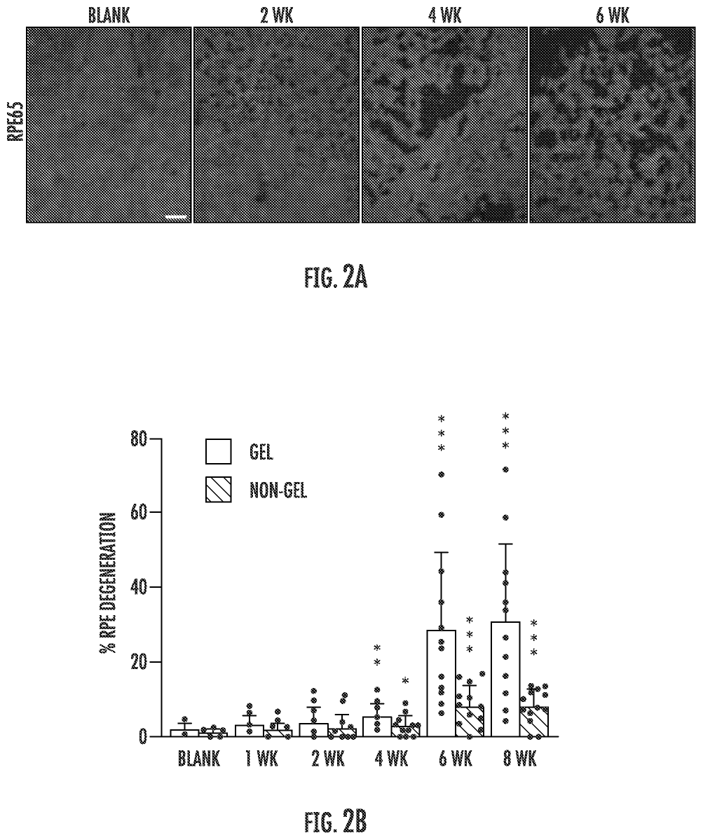

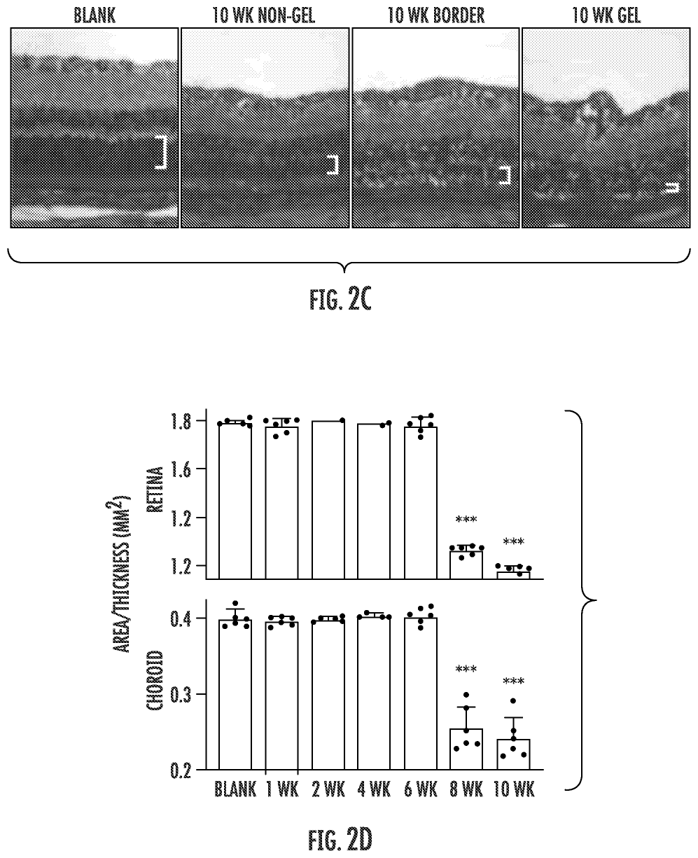

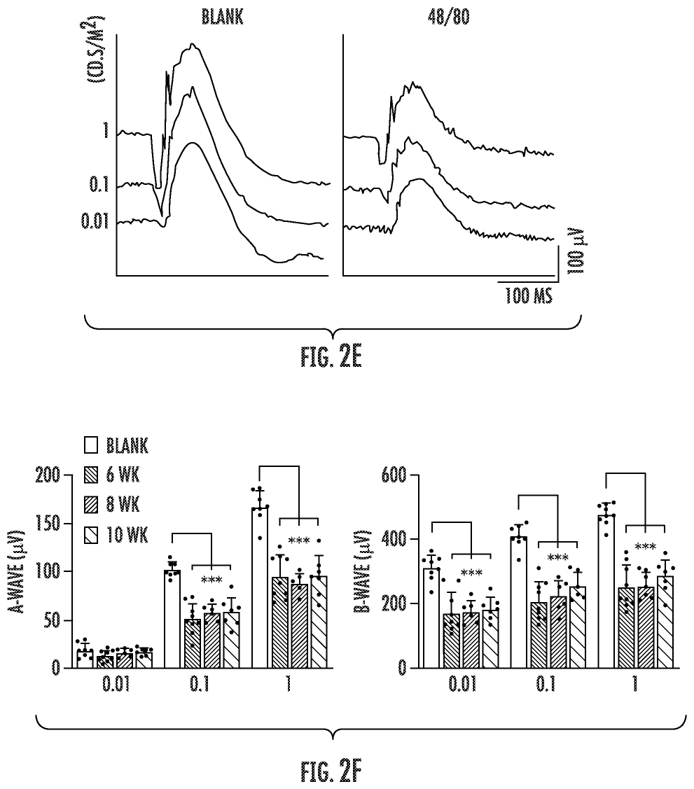

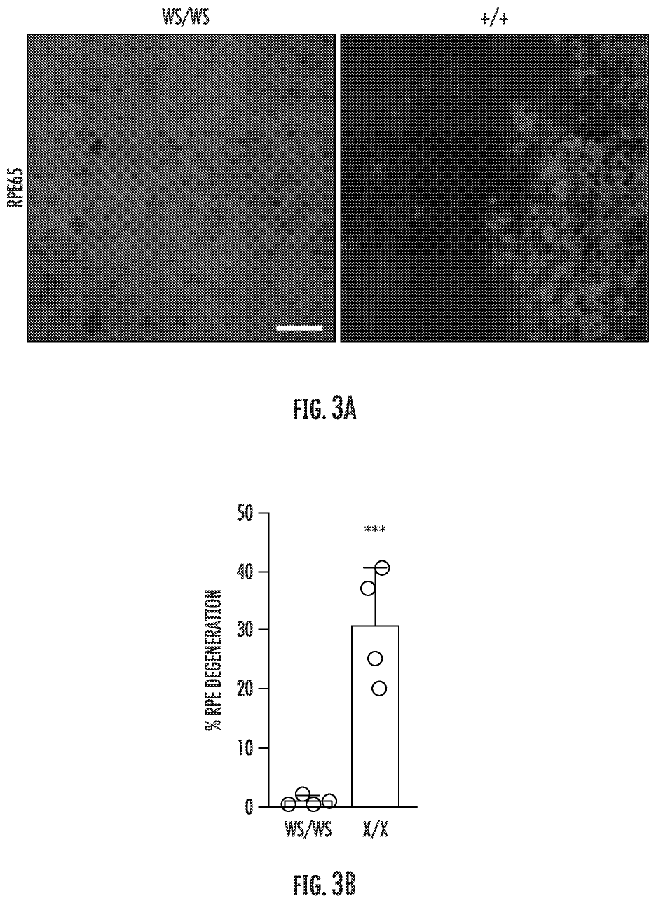

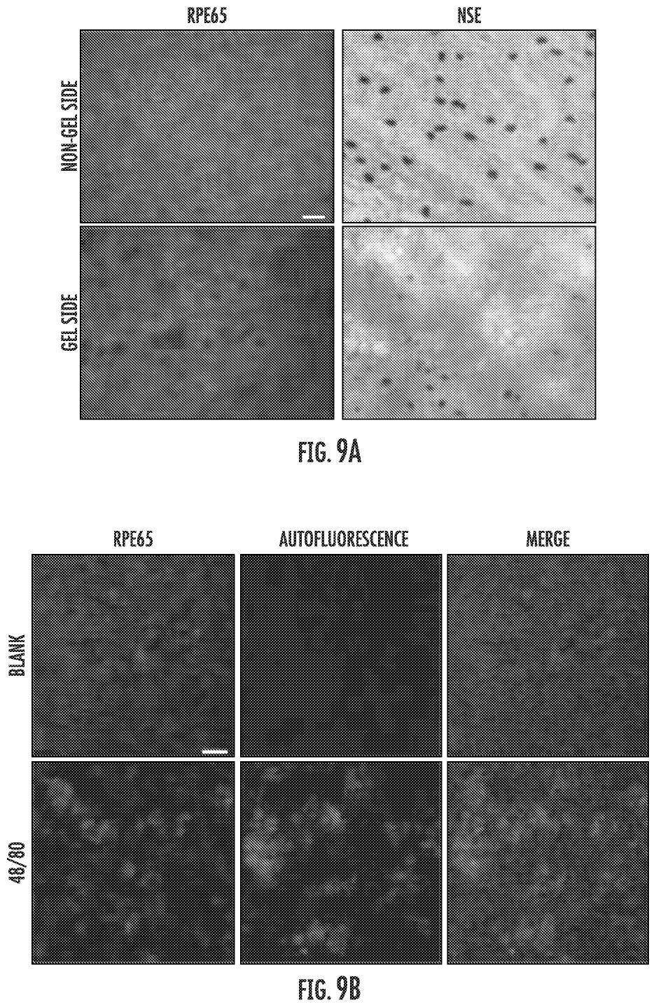

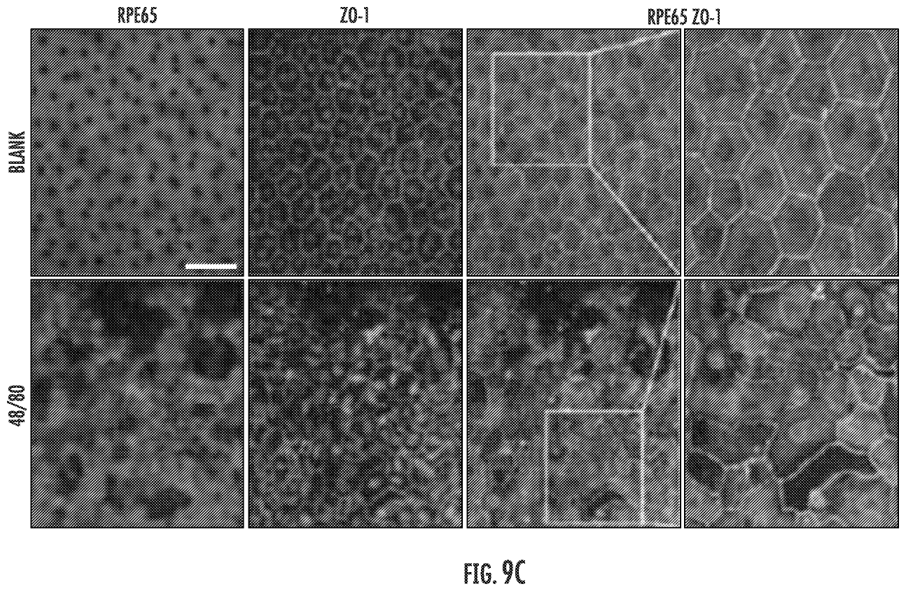

Time course after MC degranulation. RPE degeneration associated with MC degranulation was assessed next in S/D rats. Intriguingly, RPE cells stained for RPE65 in whole mount choroids showed degeneration over time following MC degranulation, with significant RPE degeneration and loss after 4 weeks (FIG. 2A,2B). Degenerating RPE cells were seen adjacent to the areas where MC degranulation was confirmed using NSE staining of the choroidal whole mounts (FIG. 9A).¹³ This indicated that RPE degeneration followed MC degranulation and there were less MCs activated and less RPE degenerated on the non-gel side (FIG. 9A). Abnormal autofluorescence of RPE cells was also detected in these lesion (FIG. 9B), consistent with human GA subjects.26 In addition, healthy hexagonal RPE cell shape was lost, RPE65 expression was greatly reduced, and there were misaligned cells suggesting tight junction deterioration, which was confirmed with co-labeling for zonula occludins-1 (ZO-1) and RPE65 (FIG. 9C). The retinas and choroids were examined histologically through the entire time course using glycol methacrylate-embedded tissue and picrosirius red stained cryosections of eyes. Following the significant RPE loss at 4 and 6 weeks post implantation, the retina and choroid demonstrated significant thinning by 8 weeks, while no significant changes were seen in the blank hydrogel implanted eyes (FIG. 2C,2D). The outer nuclear layer was thinned at 10 weeks post implantation with a gradient of severity in loss with transition from gel to the border of gel and non-gel area (FIG. 2C). This appeared to be due to photoreceptor death; therefore, visual function of the retina was assessed using scotopic ERG recordings, which revealed significant reduction in both a- and b-wave amplitude after 6 weeks (FIG. 2E,2F). Reduction of the a-wave amplitude reflects decreased photoreceptor function and the reduction in the b-wave reflects increased photoreceptor dysfunction ensuing an altered post-synaptic signal transduction cascade.²⁷ A similar reduction of scotopic ERG response in the fovea is reported in AMD patients.²⁸ Therefore, functional disturbance of the ERG at 6 weeks likely has occurred in advance of the morphological changes seen at 8 and 10 weeks post implantation.

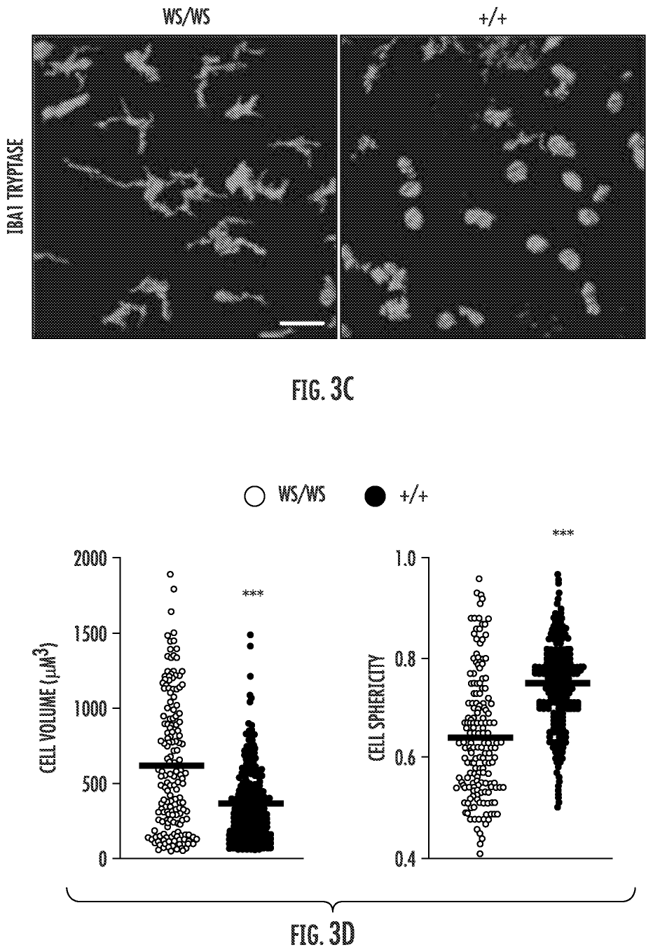

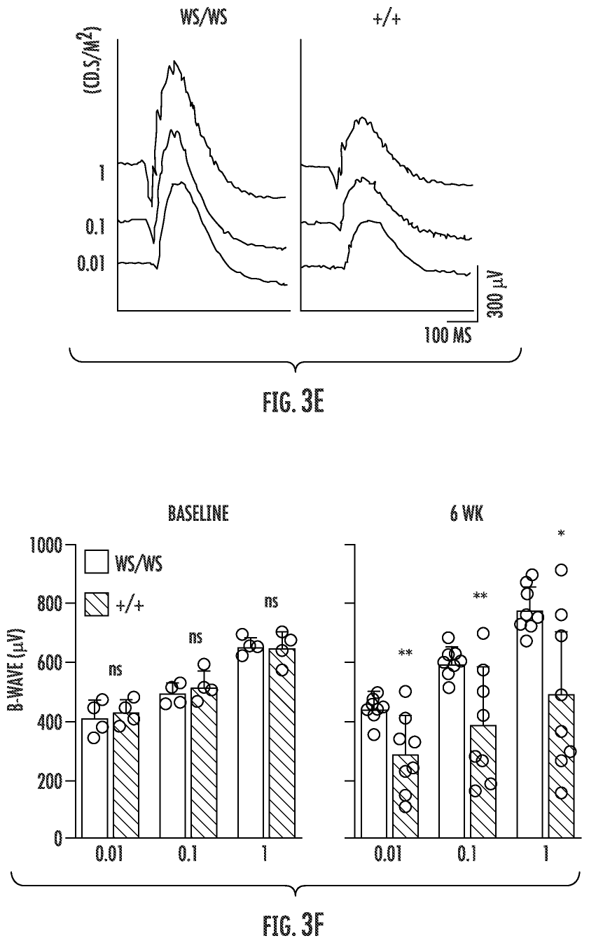

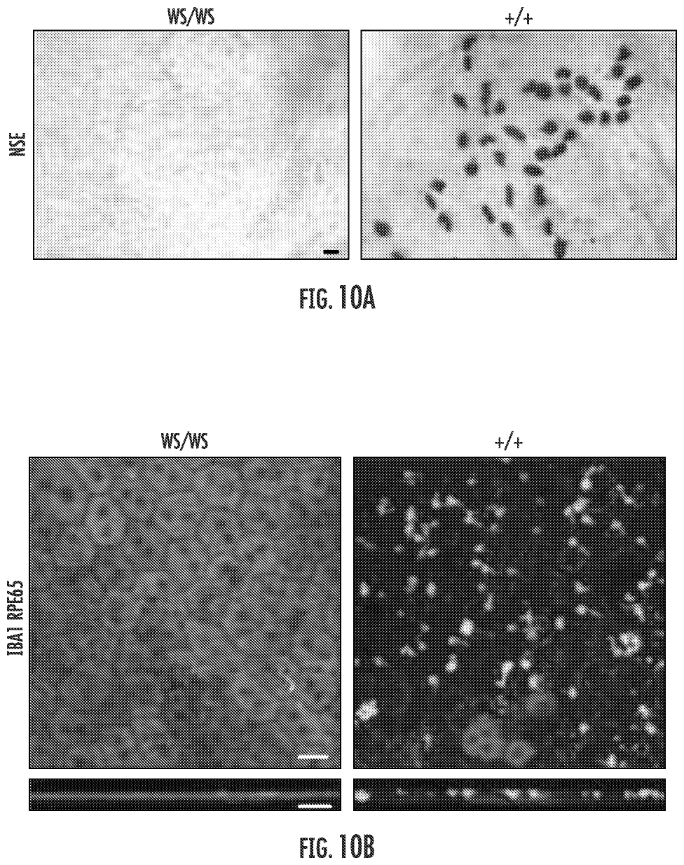

GA-like features lacking in MC deficient rats implanted with 48/80. MC deficient WsRC^{ws/ws} and their wild-type (WT) littermates (WsRC^{+/+}) were treated with subconjunctival 48/80-hydrogel to assess whether 48/80 affected only MCs and MC degranulation alone directly contributed to RPE degeneration. WsRC^{ws/ws} rats carry a defective gene for c-KIT, which is required for MC differentiation.²⁹ MCs were identified with NSE staining of the conjunctiva in WsRC^{+/+} rats but not in WsRC^{ws/ws} rats (FIG. 10A). In addition, WsRC^{ws/ws} rats showed no morphological signs of RPE degeneration at 6 weeks post 48/80-hydrogel implantation, indicating 48/80 stimulated only MCs and MC activation was responsible for RPE degeneration (FIG. 3A,3B). Choroidal macrophages were also shown to be activated in WsRC^{+/+} adjacent to MC degranulation as determined by an increase in sphericity and decrease in volume as previously reported in human GA lesion,²³ but macrophages were not activated in the MC deficient WsRC^{ws/ws} choroids (FIG. 3C,3D). The macrophages were likely activated by inflammatory cytokines released during MC degranulation and, in addition, possibly by death of adjacent RPE cells.^{23,30} Furthermore, supra-RPE and/or subretinal macrophages were detected in WsRC^{+/+} animals where RPE cells were lost, which has also been reported in human GA.³¹ However, this was not observed in WsRC^{ws/ws} rats (FIG. 10B). ERG amplitude declined in WsRC^{+/+} rats but not in WsRC^{ws/ws} rats further suggesting MC degranulation as the cause of degeneration, whereas no difference was observed between WsRC^{ws/ws} and WsRC^{+/+} rats at baseline (FIG. 3E,3F).

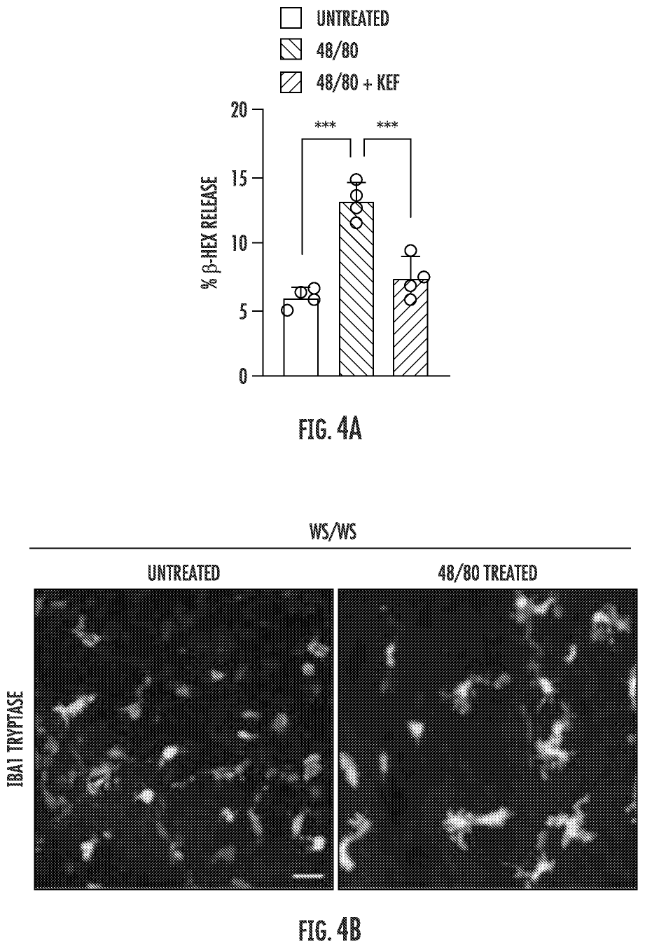

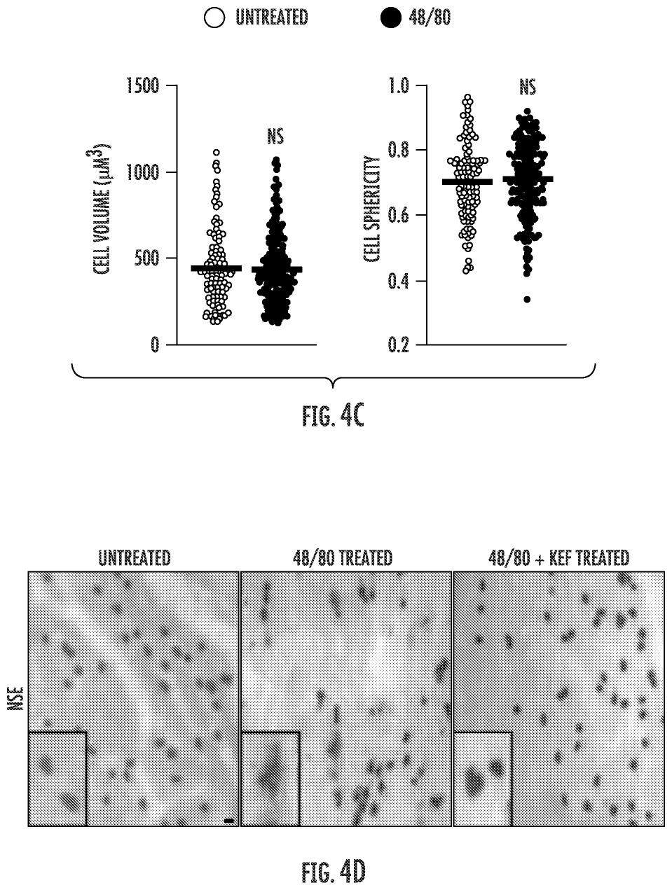

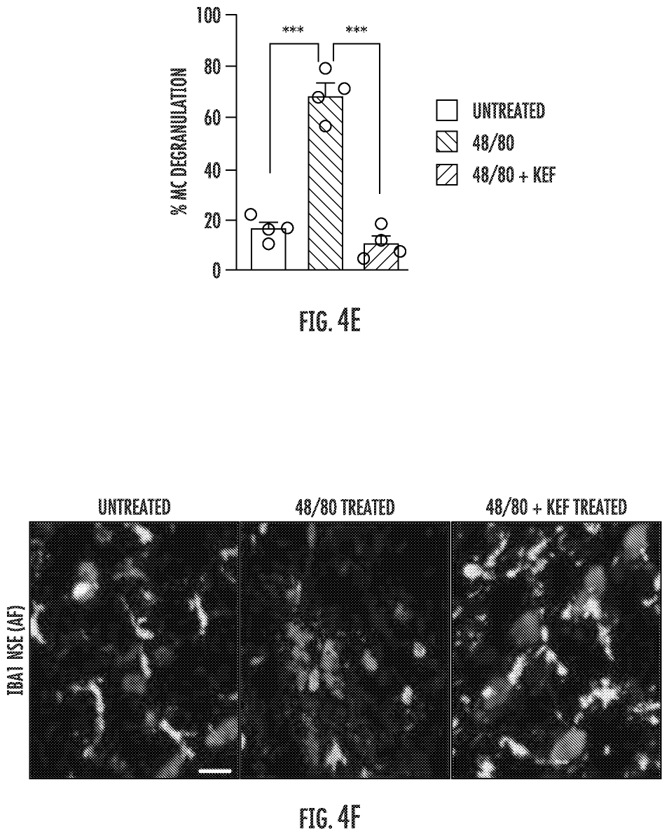

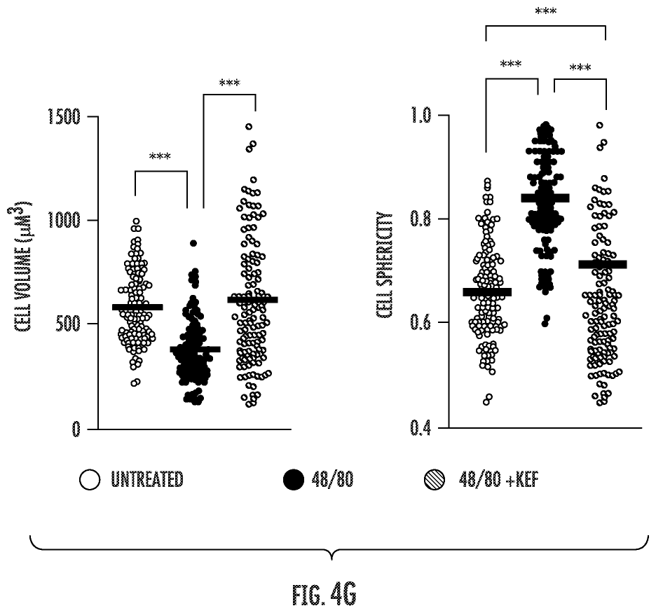

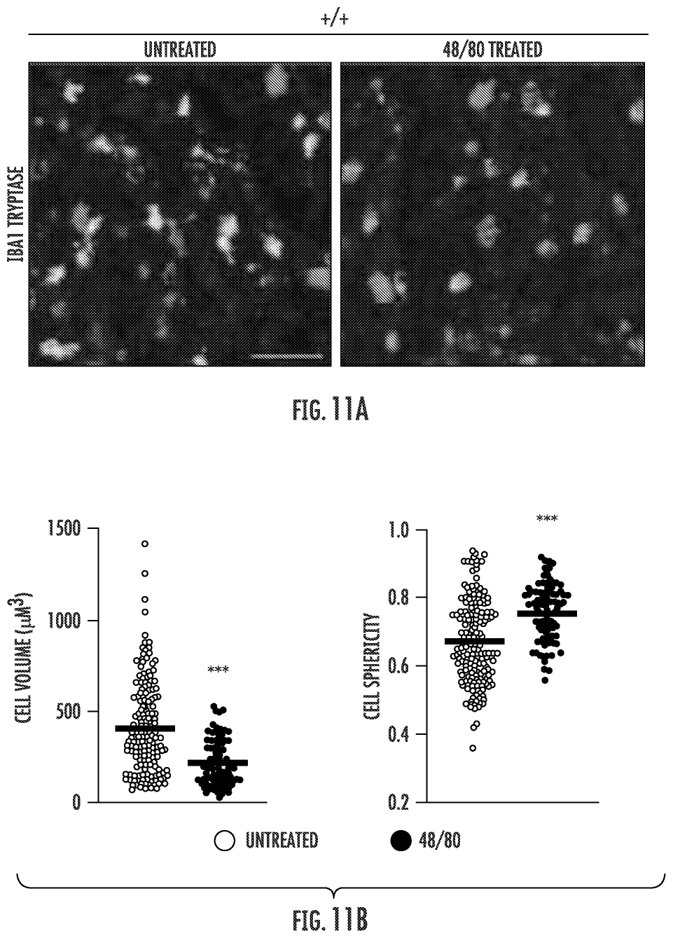

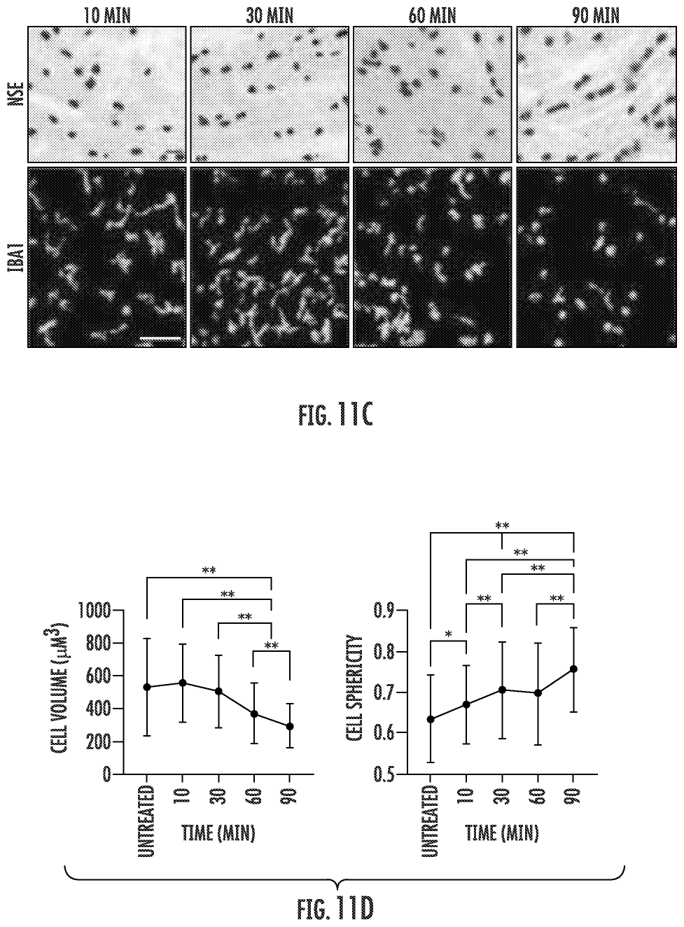

In vitro and ex vivo assays to evaluate MC degranulation and drug efficacy. Given that MCs contributed to the GA-like changes in the rat model, it appeared that MC degranulation could be a therapeutic target and, therefore, quiescing MCs might prevent this phenotype. To this end, the therapeutic potential of a MC stabilizing drug was assessed first by co-incubating the established RBL-2H3 rat MCs with ketotifen fumarate, a MC stabilizer, with or without 48/80 and analyzing the degranulation response using a β-hexosaminidase release assay, as previously reported.²² Ketotifen fumarate is a second-generation non-competitive histamine H-1 receptor antagonist. The mechanism of stabilizing MCs is assumed to be by blocking the intracellular calcium channels needed to form granules.³² It has been used to treat conditions such as asthma, allergic conjunctivitis and mastocytosis.³³⁻³⁵ Ketotifen fumarate treatment in the in vitro assay yielded a 56% inhibition of β-hexosaminidase activity (FIG. 4A). However, this cell line has shortcomings in that the cells share similarities to basophils and are derived from a basophilic leukemia.³⁶ Therefore, an acute ex vivo assay was developed to rapidly evaluate drug's efficacy and potential to stabilize choroidal MCs specifically and prevent their degranulation. After enucleation and removal of the anterior segments and retina, the eyecup from S/D rats with choroid attached was incubated for 3 hours in serum-free medium. Degranulation of MCs as well as choroidal macrophage sphericity and volume, as an indicator of macrophage activation, were evaluated in this assay. To determine that 48/80 only activated MCs ex vivo and not choroidal macrophages, MC deficient WsRC^{ws/ws} rats were utilized first in this ex vivo assay. As expected, no significant activation of macrophages was observed with or without 48/80 exposure in eyecups from WsRC^{ws/ws} rats (FIG. 4B,4C). However, littermate WsRC^{+/+} rats had significantly increased macrophage sphericity and decreased volume, indicating MC degranulation evoked macrophage activation (FIG. 11A,11B). Given that 90 minutes incubation significantly induced MC degranulation and macrophage activation (FIG. 11C,11D), the present inventors concluded that 3 hours would be sufficient to evaluate drug efficacy. After 3 hours, approximately 70% of the MCs were degranulated with 48/80 (FIG. 4D,4E) and co-incubation with ketotifen fumarate prevented MC degranulation, equivalent to the level of untreated eyecups (FIG. 4E). In addition, choroidal macrophage activation was prevented by quiescing MCs with ketotifen fumarate (FIG. 4F,4G).

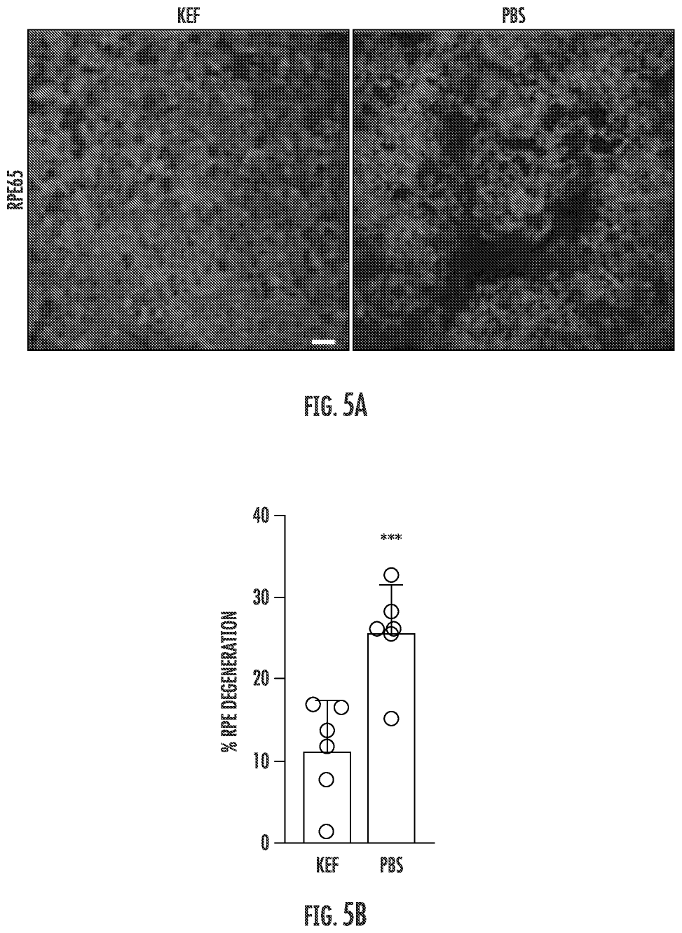

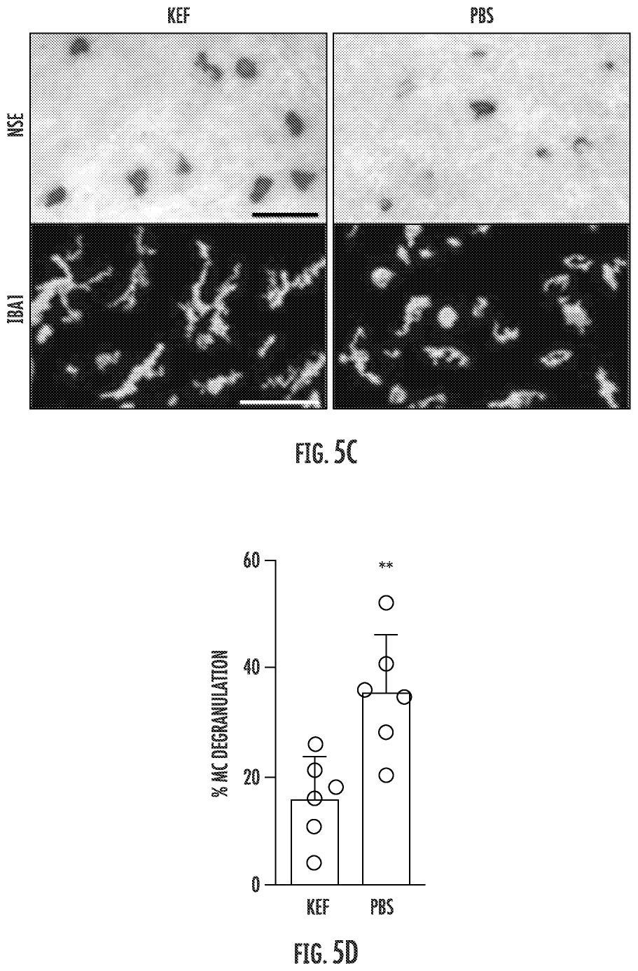

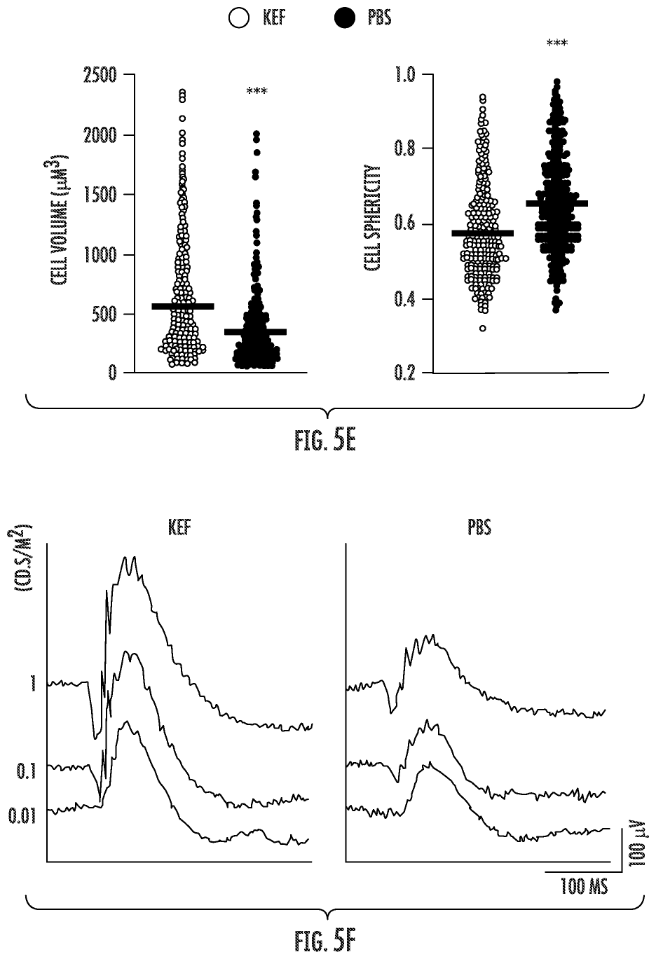

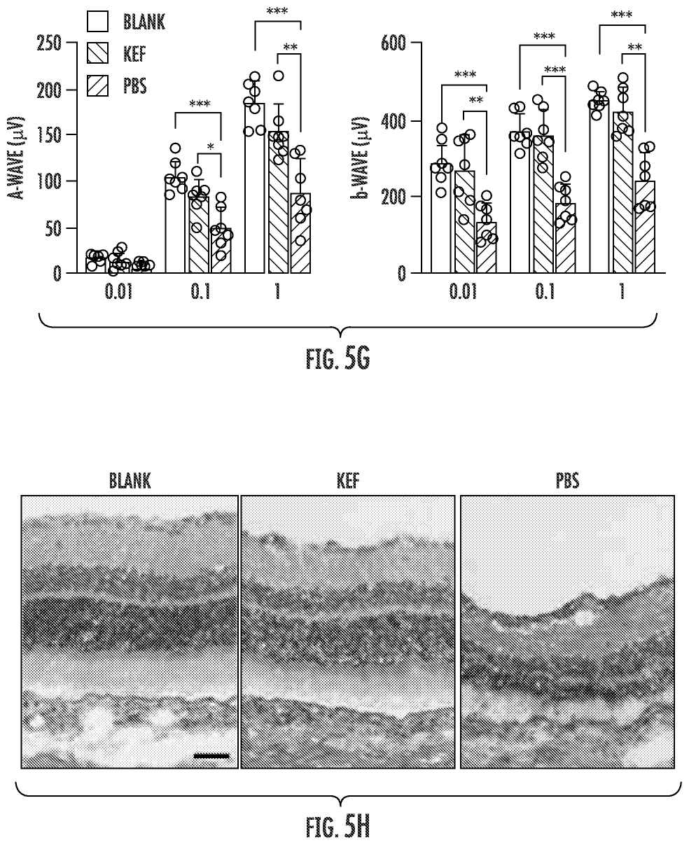

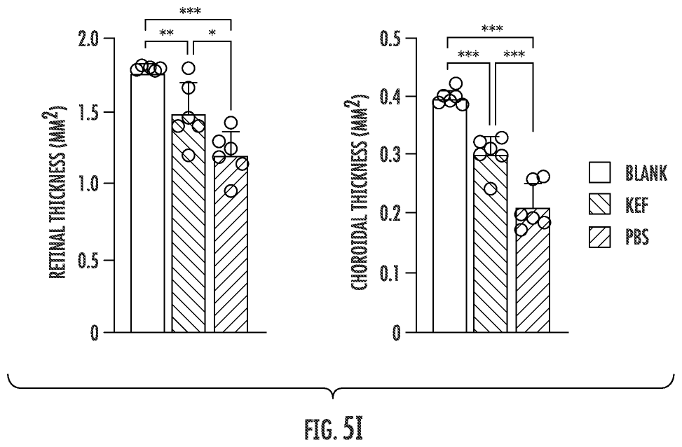

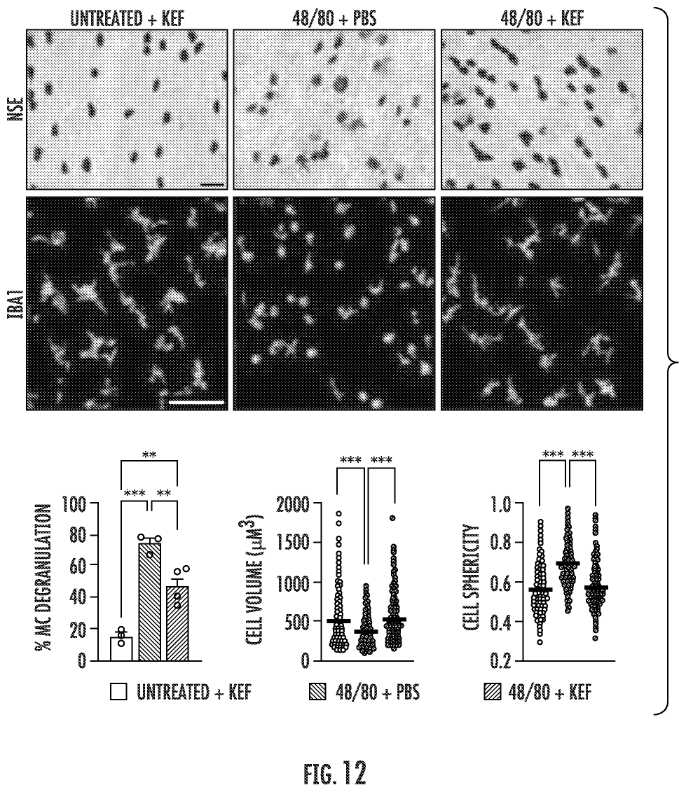

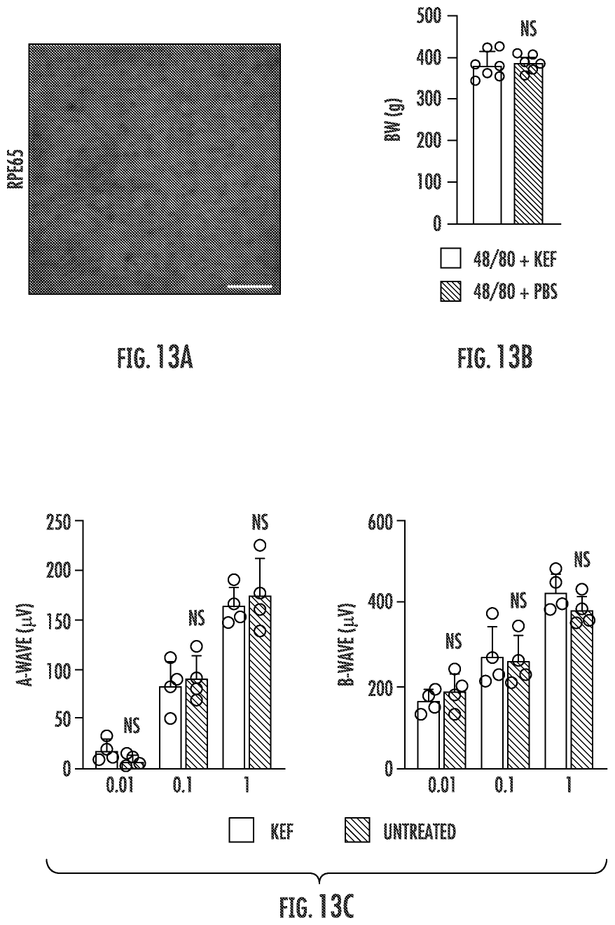

Ketotifen fumarate and tryptase inhibitor as therapeutic drugs. To assess whether ketotifen fumarate could prevent GA-like changes in vivo in the present inventors' rat model, pharmacokinetic (pK) evaluation of oral ketotifen fumarate was performed first. Rats were given 3 mg/kg of ketotifen fumarate in PBS or PBS as control once daily by gavage until sacrifice. The animals for pK were sacrificed on the 5th day and the retina and choroid were dissected from the globes. The pK evaluation indicated that there was 13-29 nM ketotifen fumarate in choroid and 12-52 nM in retina. Knowing that the drug reached choroid, 15 mg/kg of ketotifen fumarate in PBS was administered twice daily by gavage, while controls received PBS orally. Pretreatment of rats with ketotifen fumarate for 4 days prior to enucleation prevented 37% of MC degranulation and significantly prevented macrophage activation in the ex vivo assay (FIG. 12) compared to PBS treatment, indicating the potential of oral ketotifen fumarate administration in vivo. In the rat in vivo model, twice daily of oral treatment with ketotifen fumarate (15 mg/kg) significantly prevented RPE degeneration (FIG. 5A,5B) and MC degranulation (FIG. 5C,5D) at 4 weeks post 48/80-hydrogel implantation. Choroidal macrophage/monocyte activation (increased sphericity and decreased volume) was also prevented (FIG. 5C,5E). In addition, treatment with ketotifen fumarate prevented decline of the ERG amplitude in both a- and b-waves (FIG. 5F,5G). Histologic evaluation of the retina and choroidal thickness at 8 weeks post implantation demonstrated that retinochoroidal thinning was inhibited by quiescing MCs with ketotifen fumarate (FIG. 5H,5I). Treating animals with ketotifen fumarate did not affect RPE morphology or viability (FIG. 13A) nor did it affect growth of the animals as assessed by body weight measurements (FIG. 13B). ERG amplitudes were normal after 6 weeks of drug treatment alone (FIG. 13C). This indicated that administration of ketotifen fumarate orally did not manifest any adverse effects on the rats in the present inventors' study.

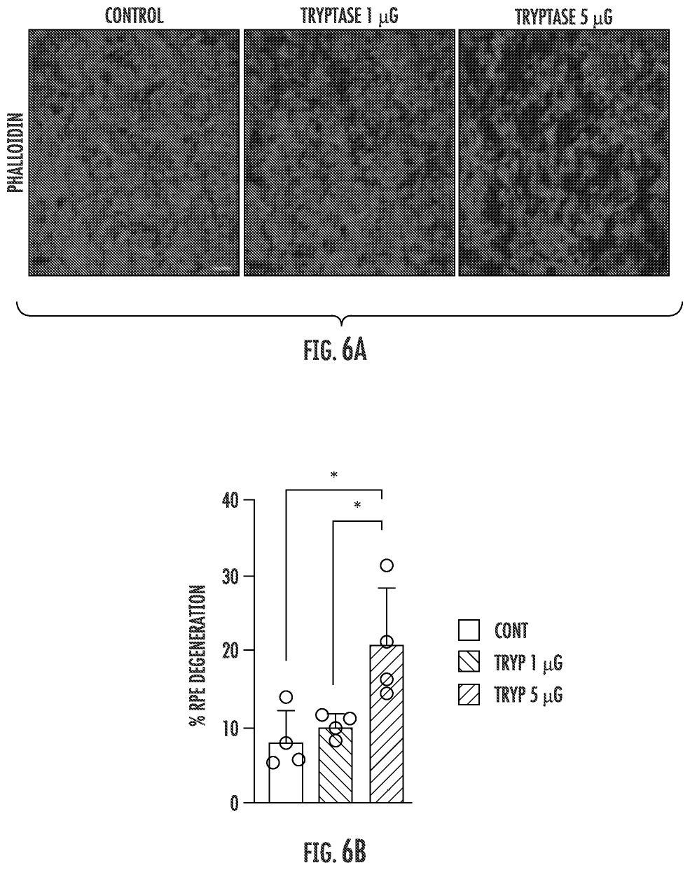

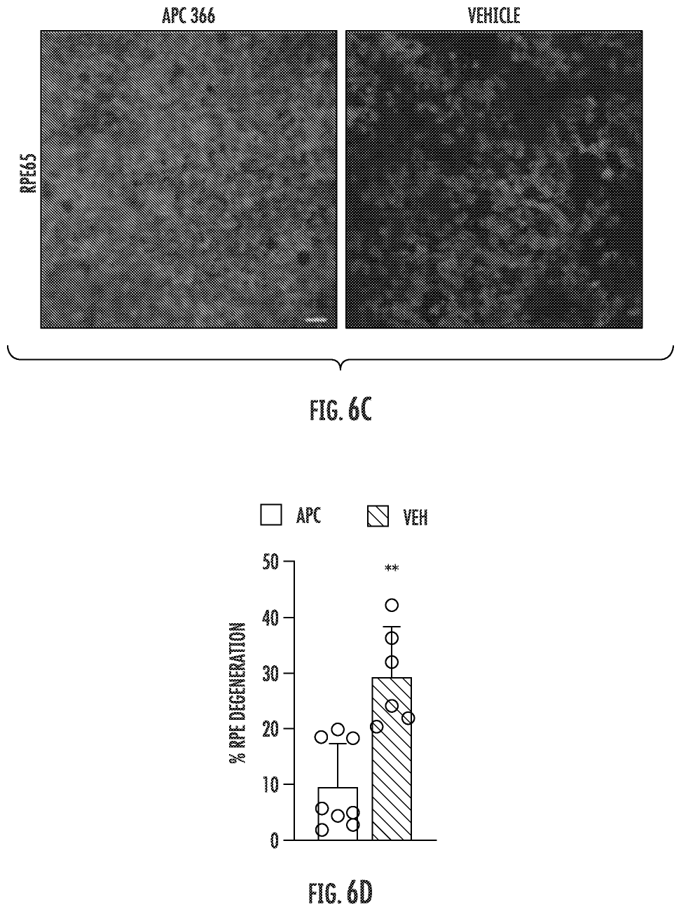

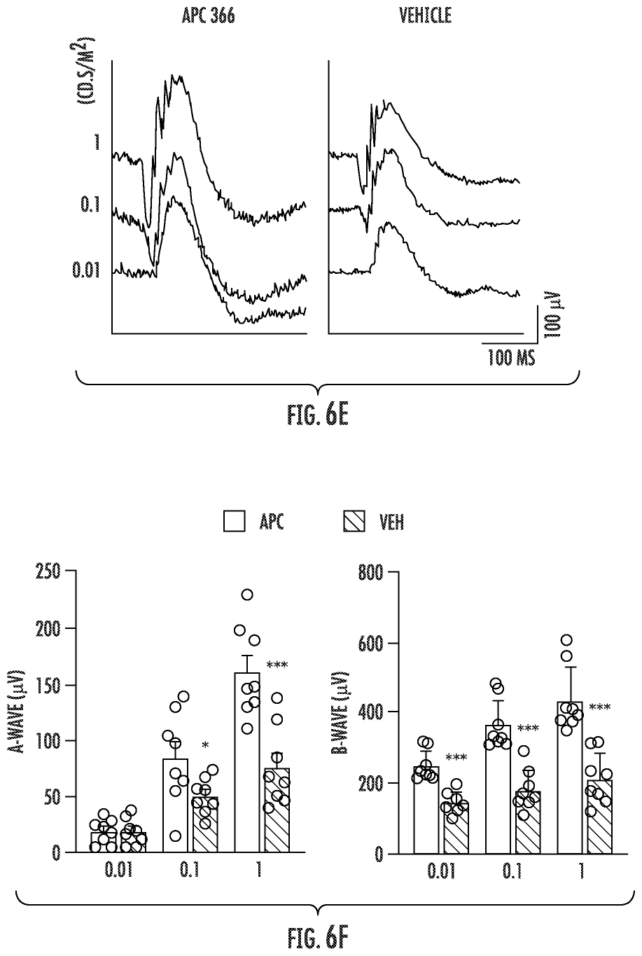

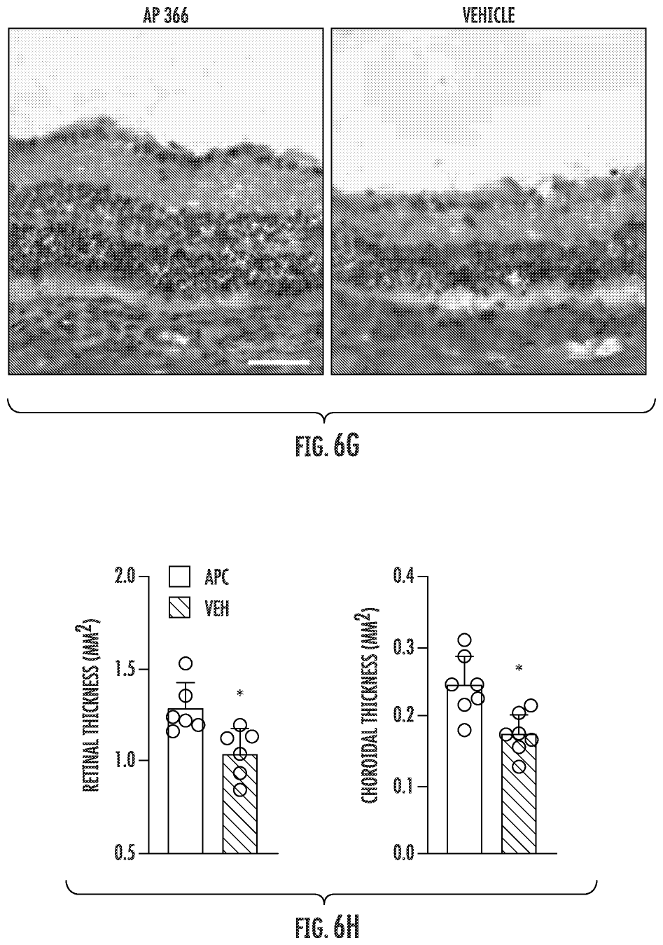

Tryptase is the most abundant protease released by MCs' and tryptase release was increased in choroid/Bruch's membrane of GA subjects.¹⁷ A similar increase in tryptase release was observed in the rat model (FIG. 1D). To test the hypothesis that tryptase directly affects RPE cell viability, in vitro assays were performed first. ARPE-19 cells were cultured with serum-free medium with or without tryptase (either 1 or 5 μg/mL). After 72 hours incubation, tryptase dose dependently induced ARPE-19 cells loss from a monolayer (FIG. 6A,6B), confirming that tryptase can affect RPE adherence and cell viability.

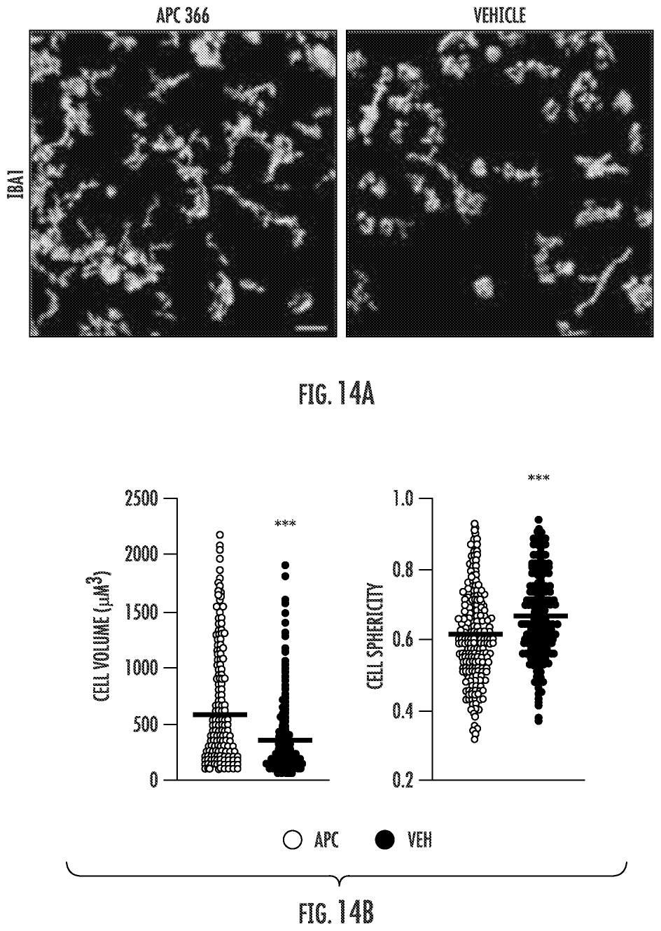

Therefore, the tryptase inhibitor APC 366, which is reported to prevent allergic, inflammatory or fibrotic changes in human and animal models^{24,38,39} was evaluated in the present inventors' 48/80-hydrogel implant rat model. Rats were given a subcutaneous injection of APC 366 (5 mg/kg) or vehicle daily after 48/80 implantation until sacrifice. After 4 weeks treatment, RPE degeneration was significantly inhibited (FIG. 6C,7D) and choroidal macrophage/monocyte activation was prevented (FIG. 14). Furthermore, reduction in ERG amplitude of both a- and b-waves was prevented at 8 weeks (FIG. 6E,6F). When retinal and choroidal thickness were evaluated, APC 366 treated animals had significantly less retinochoroidal thinning (FIG. 6G,6H); therefore, all the GA-like changes were suppressed. Collectively, these findings suggest that MC-derived tryptase is a key molecule in development of GA-like changes following chronic MC degranulation.

### Discussion

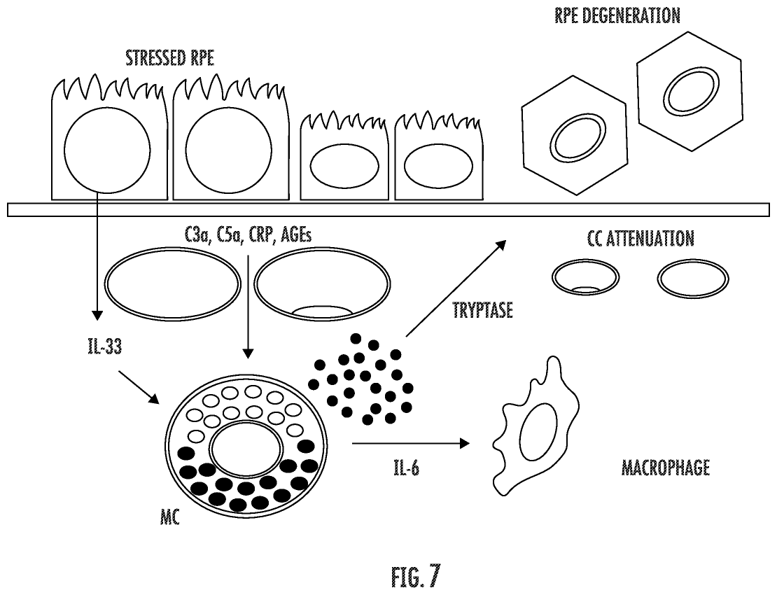

GA is a multifactorial disease characterized by RPE loss and choriocapillaris attenuation and retinal and choroidal atrophy that is clinically manifested over many years. There is increasing evidence that hypoxia-initiated oxidative stress and inflammation contribute to GA pathogenesis.⁴⁰ Nevertheless, there is insufficient understanding of the cellular or molecular mechanism underlying the development of GA, as well as, few animal models reproducing a GA-like phenotype.¹² This paucity of knowledge and animal models has hampered the drug development and targeting. In the current study, the present inventors present evidence that chronic degranulation of choroidal MCs can induce many phenotypic changes in retina and choroid that occur in GA: RPE degeneration, visual function decline, and retinal and choroidal thinning. However, another hallmark of GA, attenuation of choriocapillaris,²¹ did not occur in the present inventors' model, perhaps due to the regenerative capability of the rodent choriocapillaris.⁴¹ Furthermore, the present inventors developed both ex vivo and in vivo models, which demonstrate that choroidal MC degranulation alone can stimulate choroidal macrophage activation as observed in human GA.²³ Using these models, the present inventors demonstrated that MC stabilization and tryptase inhibition can prevent these MC-induced GA-like phenotypes. MCs are the initial responders of innate immunity and their abundance in the uveal tract and choroid suggest their fundamental importance in the eye.¹ Indeed, they respond to toxins and microbes as well as substances found in GA choroid such as AGEs, C3a, C5a, and CRP, all of which are implicated in AMD.^{14,15,42,43} Recently, the present inventors reported that choroidal MCs were increased in number and their degranulation was increased in GA subjects.¹³ The present inventors do not know exactly when abnormal MC degranulation occurs in GA choroid, but in that study the present inventors found a significant increase in MC numbers and number of degranulated in early AMD subjects, suggesting it is occurring early in the disease process. In addition, MC-derived tryptase was localized in Bruch's membrane.^{13,17} Nevertheless, the precise role of MCs in the development of GA had yet to be elucidated. Here, the present inventors demonstrate that hydrogel containing 48/80 implanted subconjunctivally resulted in a gradual and protracted activation and degranulation of choroidal MCs in the rat. Continuous MC degranulation mediated chronic macrophage activation and induced RPE degeneration with subsequent retina and choroidal thinning, four characteristics of human GA.^{21,23} The present inventors further show evidence that MC-derived tryptase, at least in part, through degradation of the choroid stroma and Bruch's membrane, contributed to RPE degeneration. The pathological phenotype was prevented by genetically eliminating MCs and by pharmacological intervention targeting MCs with a generic MC stabilizer ketotifen fumarate and a MC specific tryptase inhibitor APC 366. Therefore, MCs with aberrant degranulation and specifically MC-derived tryptase appear to play a central role in developing a GA-like pathology. Therefore, the present inventors propose the hypothesis that choroidal MC degranulation and release of MC-derived tryptase (FIG. 7), results in degradation of Bruch's membrane and choroidal stroma, which ultimately progresses to RPE degeneration as well as retinal and choroidal thinning, which are hallmarks of GA.

To evaluate the efficacy of targeting MCs in GA, development of an assay to screen potential therapeutic compounds was deemed indispensable. To this end, the present inventors utilized the well-established rat MC line, RBL-2H3 cells, and their production of β-hexosaminidase, an MC granule enzyme.

Although RBL-2H3 cells have been broadly used to assess MC degranulation and drug screening, various contradictions and limitations have also been reported.³⁶ There remains controversy in the lineage of RBL-2H3 cells in that they resemble basophils rather than MCs, which is highly likely because these cells originate from a basophilic leukemia.³⁶ The ex vivo assay developed for these studies overcame those limitations and provided a rapid assay in a tissue milieu involved in AMD, the choroid and its inflammatory cells. This assay enabled sufficient choroidal MC degranulation and effective drug screening in 3 hours and yielded data on three important parameters: percentage of MCs degranulated in choroid, and volume and sphericity of choroidal macrophages, indicating macrophage activation. The lack of blood flow enabled direct evaluation of only choroidal MCs and resident macrophages and not interaction with circulating cells. In order to exclude the possibility of 48/80 influencing macrophages directly and not activating MCs specifically, MC deficient WsRC^{WS/WS} and their WT control rats were first evaluated in this ex vivo assay. Choroidal macrophages were only activated in WT, not in MC deficient rat choroids, suggesting cytokines released after MC activation and degranulation-induced macrophage activation. Similar findings were confirmed in vivo, collectively indicating that cytokines released from activated MCs induced focal inflammation and contributed at least in part to the disease progression. However, the present inventors realize that the present inventors' model only represents MC interaction with macrophages and macrophages in AMD choroid are exposed to an activated complement cascade and CRP as well as MC granule contents.

In the current study, the pharmaceutical benefit of ketotifen fumarate, an FDA-approved generic MC stabilizer, was evaluated in vitro, ex vivo, and then, in vivo, where it prevented MC degranulation efficiently in all experiments. One of the major complications reported with oral daily treatment was drowsiness but this is said to decrease with time.³³ Patients with mastocytosis and chronic asthma are managed chronically with oral MC stabilizers like ketotifen fumarate without severe complications, suggesting that the drug is rather innocuous.^{44,45} However, a recent report demonstrated that ketotifen fumarate dose dependently decreased non-rapid eye movement (REM) sleep and increased REM sleep in rats; therefore, it might function as an aid for desirable sleep.⁴⁶ Nevertheless, no adverse effects with oral ketotifen fumarate treatments were observed in the present inventors' study.

Inhibiting MC-derived tryptase activity with APC 366 successfully inhibited all GA-like changes in the present inventors' rat model from RPE degeneration to macrophage activation after MC degranulation. Degeneration of RPE and retinal and choroidal thinning was expected because tryptase degrades collagens and activates MMPs, which further degrade stroma and basement membranes. Previous reports have shown that tryptase released via MC degranulation amplified the production of intercellular adhesion molecule-1, CC-motif ligand 2, and IL-8 in endothelial and epithelial cells.^{47,48} Most recently, treating mice with APC 366 suppressed inflammation as assessed by the transcriptional expression of multiple mediators including IL-1β, IL-6, IL-8, CC-motif ligand 2, and MMP-3 in an osteoarthritis model.⁴⁹ Thus, the effect of tryptase released in degranulation resulting in macrophage activation might well be expected in the present inventors' model. Also, MCs release IL-6 and tumor necrosis factor when activated, further stimulating macrophage activation.⁴⁹ Taken together, monocyte/macrophage activation in the choroid and activation of resident macrophages was presumably prevented by inhibiting tryptase with APC 366, hence inhibiting focal inflammation. The present inventors have clarified a critical role of MCs in RPE degeneration and the presence of MC-derived pro-inflammatory cytokines and proteases can be presumed to contribute to disease development based on other studies.^{38,47} One scenario might be that MCs are activated by stressed RPE pro-inflammatory molecules in the GA choroid and then, catalyze tissue destruction via tryptase and also trigger inflammation and macrophage activation, which facilitates the vicious cycle of degeneration in the GA photoreceptor/RPE/Bruch's membrane/choriocapillaris complex (FIG. 7). The present inventors' model (FIG. 7) is obviously a simplified scenario in which the effects of mast cell degranulation are highlighted. The present inventors' model does not address the complexities of human GA where the complement cascade is activated,⁵⁰ perhaps in the presence of a defective complement factor H, complement can then activate choroidal macrophages,²³ and oxidative stress is known to play a key role.⁴⁰ Even with those challenges lacking, tissue destruction and a GA phenotype resulted in the model, suggesting that MCs could initiate or at least contribute to GA. Alternatively, MC degranulation like complement activation could be a result of AMD and not the cause. In AMD choroid, complement is activated (elevated C3a and C5a),¹⁴ Complement factor H declines and CRP is greatly elevated,¹⁵ providing three potential stimuli for MC migration and degranulation.

In conclusion, this is the first study to demonstrate the involvement of MC degranulation and release of tryptase in initiating GA-like pathologic changes in the eye. It would be interesting in the future to investigate if there is any relationship between incidence of GA and MC activating diseases like mastocytosis, MC activating syndrome, Ehlers-Danos syndrome, and fibromyalgia. The etiology of RPE degeneration and choroidal thinning in GA was not known prior to this study. The present inventors' model suggests a potential role for MCs in GA without addressing the pro-inflammatory milieu of GA RPE/choroid complex, where complement is activated and oxidative stress is present. A MC stabilizer or inhibition of MC-derived tryptase prevented the disease onset and progression in the rat model. These results support the novel concept that MCs contribute to the development of GA and choroidal MCs could be a viable therapeutic target for the currently untreatable GA.

### REFERENCES

1. McMenamin P G. The distribution of immune cells in the uveal tract of the normal eye. Eye. 1997; 11:183-193.
2. Wernersson S, Pejler G. Mast cell secretory granules: armed for battle. Nat Rev Immunol. 2014; 14:478-494.
3. Schwartz L B. Mediators of human mast cells and human mast cell subsets. Ann Allergy. 1987; 58:226-235.
4. Iddamalgoda A, Le Q T, Ito K, Tanaka K, Kojima H, Kido H. Mast cell tryptase and photoaging: possible involvement in the degradation of extra cellular matrix and basement membrane proteins. Arch Dermatol Res. 2008; 300(suppl. 1):S69-S76.
5. Bradding P, Walls A F, Holgate S T. The role of the mast cell in the pathophysiology of asthma. J Allergy Clin Immunol. 2006; 117:1277-1284.
6. Ponomaryov T, Payne H, Fabritz L, Wagner D D, Brill A. Mast cells granular contents are crucial for deep vein thrombosis in mice. Circ Res. 2017; 121:941-950.
7. Sun J, Sukhova G K, Yang M, et al. Mast cells modulate the pathogenesis of elastase-induced abdominal aortic aneurysms in mice. J Clin Investig. 2007; 117:3359-3368.
8. Krystel-Whittemore M, Dileepan K N, Wood J G. Mast cell: a multi-functional master cell. Front Immunol. 2015; 6:620.
9. Godfrey W A. Characterization of the choroidal mast cell. Trans Am Ophthalmol Soc. 1987; 85:557-599.
10. Wong W L, Su X, Li X, et al. Global prevalence of age-related macular degeneration and disease burden projection for 2020 and 2040: a systematic review and meta-analysis. Lancet Glob Health. 2014; 2:e106-e116.
11. Holz F G, Strauss E C, Schmitz-Valckenberg S, van Lookeren Campagne M. Geographic atrophy: clinical features and potential therapeutic approaches. Ophthalmology. 2014; 121:1079-1091.
12. Pennesi M E, Neuringer M, Courtney R J. Animal models of age related macular degeneration. Mol Aspects Med. 2012; 33:487-509.
13. Bhutto I A, McLeod D S, Jing T, Sunness J S, Seddon J M, Lutty G A. Increased choroidal mast cells and their degranulation in age-related macular degeneration. Br J Ophthalmol. 2016; 100:720-726.
14. Nozaki M, Raisler B J, Sakurai E, et al. Drusen complement components C3a and C5a promote choroidal neovascularization. Proc Natl Acad Sci USA. 2006; 103:2328-2333.
15. Bhutto I A, Baba T, Merges C, Juriasinghani V, McLeod D S, Lutty G A. C-reactive protein and complement factor H in aged human eyes and eyes with age-related macular degeneration. Br J Ophthalmol. 2011; 95:1323-1330.
16. Yamada Y, Ishibashi K, Ishibashi K, et al. The expression of advanced glycation endproduct receptors in RPE cells associated with basal deposits in human maculas. Exp Eye Res. 2006; 82:840-848.
17. McLeod D S, Bhutto I, Edwards M M, Gedam M, Baldeosingh R, Lutty G A. Mast cell-derived tryptase in geographic atrophy. Invest Ophthalmol Vis Sci. 2017; 58:5887-5896.
18. Payne V, Kam P C. Mast cell tryptase: a review of its physiology and clinical significance. Anaesthesia. 2004; 59:695-703.
19. Bhutto I A, Ogura S, Baldeosingh R, McLeod D S, Lutty G A, Edwards M M. An acute injury model for the phenotypic characteristics of geographic atrophy. Invest Opthalmol Vis Sci. 2018; 59:AMD143-AMD151.
20. Soiberman U, Kambhampati S P, Wu T, et al. Subconjunctival injectable dendrimer-dexamethasone gel for the treatment of corneal inflammation. Biomaterials. 2017; 125:38-53.
21. McLeod D S, Grebe R, Bhutto I, Merges C, Baba T, Lutty G A. Relationship between RPE and choriocapillaris in age-related macular degeneration. Invest Ophthalmol Vis Sci. 2009; 50:4982-4991.
22. Weatherly L M, Kennedy R H, Shim J, Gosse J A. A microplate assay to assess chemical effects on RBL-2H3 mast cell degranulation: effects of triclosan without use of an organic solvent. J Vis Exp. 2013; 81:e50671.
23. McLeod D S, Bhutto I, Edwards M M, Silver R E, Seddon J M, Lutty G A. Distribution and quantification of choroidal macrophages in human eyes with age-related macular degeneration. Invest Ophthalmol Vis Sci. 2016; 57:5843-5855.
24. Lu J, Chen B, Li S, Sun Q. Tryptase inhibitor APC 366 prevents hepatic fibrosis by inhibiting collagen synthesis induced by tryptase/protease-activated receptor 2 interactions in hepatic stellate cells. Int Immunopharmacol. 2014; 20:352-357.
25. Bousquet E, Zhao M, Thillaye-Goldenberg B, et al. Choroidal mast cells in retinal pathology: a potential target for intervention. Am J Pathol. 2015; 185:2083-2095.
26. Delori F C, Fleckner M R, Goger D G, Weiter J J, Dorey C K. Autofluorescence distribution associated with drusen in agerelated macular degeneration. Invest Ophthalmol Vis Sci. 2000; 41: 496-504.
27. Pardue M T, Peachey N S. Mouse b-wave mutants. Doc Ophthalmol. 2014; 128:77-89.
28. Walter P, Widder R A, Luke C, Konigsfeld P, Brunner R. Electrophysiological abnormalities in age-related macular degeneration. Graefes Arch Clin Exp Ophthalmol. 1999; 237:962-968.
29. Niwa Y, Kasugai T, Ohno K, et al. Anemia and mast cell depletion in mutant rats that are homozygous at “white spotting (Ws)” locus. Blood. 1991; 78:1936-1941.
30. Omri S, Behar-Cohen F, de Kozak Y, et al. Microglia/macrophages migrate through retinal epithelium barrier by a transcellular route in diabetic retinopathy: role of PKCzeta in the Goto Kakizaki rat model. Am J Pathol. 2011; 179:942-953.
31. Guillonneau X, Eandi C M, Paques M, Sahel J A, Sapieha P, Sennlaub F. On phagocytes and macular degeneration. Prog Retin Eye Res. 2017; 61:98-128.
32. Craps L P, Ney U M. Ketotifen: current views on its mechanism of action and their therapeutic implications. Respiration. 1984; 45:411-421.
33. Grant S M, Goa K L, Fitton A, Sorkin E M. Ketotifen. A review of its pharmacodynamic and pharmacokinetic properties, and therapeutic use in asthma and allergic disorders. Drugs. 1990; 40:412-448.
34. Kidd M, McKenzie S H, Steven I, Cooper C, Lanz R, Australian Ketotifen Study Group. Efficacy and safety of ketotifen eye drops in the treatment of seasonal allergic conjunctivitis. Br J Ophthalmol. 2003; 87:1206-1211.
35. Valent P, Sperr W R, Schwartz L B, Horny H P. Diagnosis and classification of mast cell proliferative disorders: delineation from immunologic diseases and non-mast cell hematopoietic neoplasms. J Allergy Clin Immunol. 2004; 114:3-11; quiz 12.
36. Passante E, Frankish N. The RBL-2H3 cell line: its provenance and suitability as a model for the mast cell. Inflamm Res. 2009; 58:737-745.
37. Schwartz L B, Irani A M, Roller K, Castells M C, Schechter N M. Quantitation of histamine, tryptase, and chymase in dispersed human T and T C mast cells. J Immunol. 1987; 138:2611-2615.
38. Wang Q, Lepus C M, Raghu H, et al. IgE-mediated mast cell activation promotes inflammation and cartilage destruction in osteoarthritis. eLife. 2019; 8:e39905.
39. Krishna M T, Chauhan A, Little L, et al. Inhibition of mast cell tryptase by inhaled APC 366 attenuates allergen-induced late-phase airway obstruction in asthma. J Allergy Clin Immunol. 2001; 107:1039-1045.
40. Datta S, Cano M, Ebrahimi K, Wang L, Handa J T. The impact of oxidative stress and inflammation on RPE degeneration in non-neovascular AMD. Prog Retin Eye Res. 2017; 60:201-218.
41. Hayashi A, Majji A B, Fujioka S, et al. Surgically induced degeneration and regeneration of the choriocapillaris in rabbit. Graefes Arch Clin Exp Ophthalmol. 1999; 237:668-677.
42. Hartmann K, Henz B M, Kruger-Krasagakes S, et al. C3a and C5a stimulate chemotaxis of human mast cells. Blood. 1997; 89:2863-2870.
43. Yamagishi S, Fujimori H, Yonekura H, Yamamoto Y, Yamamotos H. Advanced glycation endproducts inhibit prostacyclin production and induce plasminogen activator inhibitor-1 in human microvascular endothelial cells. Diabetologia. 1998; 41:1435-1441.
44. Kabra S K, Pandey R M, Singh R, Seth V. Ketotifen for asthma in children aged 5 to 15 years: a randomized placebo-controlled trial. Ann Allergy Asthma Immunol. 2000; 85:46-52.
45. Marone G, Spadaro G, Granata F, Triggiani M. Treatment of mastocytosis:
   pharmacologic basis and current concepts. Leuk Res. 2001; 25:583-594.
46. Unno K, Ozaki T, Mohammad S, et al. First and second generation H(1) histamine receptor antagonists produce different sleep-inducing profiles in rats. Eur Pharmacol. 2012; 683: 179-185.
47. Kinoshita M, Okada M, Hara M, Furukawa Y, Matsumori A. Mast cell tryptase in mast cell granules enhances MCP-1 and interleukin-8 production in human endothelial cells. Arterioscler Thromb Vasc Biol. 2005; 25:1858-1863.
48. Cairns J A, Walls A F. Mast cell tryptase is a mitogen for epithelial cells. Stimulation of I L-8 production and intercellular adhesion molecule-1 expression. J Immunol. 1996; 156:275-283.
49. Theoharides T C, Alysandratos K D, Angelidou A, et al. Mast cells and inflammation. Biochim Biophys Acta. 2012; 1822:21-33.
50. Kauppinen A, Paterno J J, Blasiak J, Salminen A, Kaarniranta K. Inflammation and its role in age-related macular degeneration. Cell Mol Life Sci. 2016; 73:1765-1786.

1. A method comprising the steps of:
(a) administering a drug to a mammal, wherein the mammal comprises a rat or a mouse;
(b) enucleating the eyes of the mammal;
(c) removing the anterior eye and excising the retina from the eye, wherein the eye comprises an eyecup that comprises choroidal mast cells (MCs); and
(d) measuring mast cell degranulation.

2. The method of claim 1, wherein administering step (a) further comprises administering an agent that promotes MC degranulation.

3. The method of claim 2, wherein the agent comprises compound 48/80 or calcium ionophore.

4. The method of claim 1, wherein step (d) comprises staining for non-specific esterase (NSE) activity in mast cells present in the choroid of the eyecup.

5. The method of claim 4, further comprising counting non-degranulated and granulated cells in the whole choroid, wherein degranulated cells exhibit irregular shape or extracellular granules.

6. The method of claim 1, further comprising staining macrophages and measuring macrophage cell volume and sphericity.

7. The method of claim 1, wherein macrophages are stained with anti-Iba1 antibody.

8. The method of claim 1, further comprising staining for tryptase with an anti-MC tryptase antibody.

9. A method comprising the steps of:
(a) contacting an eyecup of a mammal with a drug, wherein the eyecup comprises choroidal mast cells; and
(b) measuring MC degranulation

10. The method of claim 9, wherein the eyecup has been separated from the anterior eye and retina.

11. The method of claim 9, wherein contacting step (a) further comprises contacting the eyecup with an agent that promotes MC degranulation.

12. The method of claim 11, wherein the agent comprises compound 48/80 or calcium ionophore.

13. The method of claim 9, wherein step (b) comprises staining for NSE activity in mast cells present in the choroid of the eyecup.

14. The method of claim 13, further comprising counting non-degranulated and granulated cells in the whole choroid, wherein degranulated cells exhibit irregular shape or extracellular granules.

15. The method of claim 9, wherein the method further comprises staining macrophages and measuring macrophage cell volume and sphericity.

16. The method of claim 15, wherein macrophages are stained with anti-Iba1 antibody.

17. The method of claim 9, further comprising staining for tryptase with an anti-MC tryptase antibody.

18. The method of claim 9, wherein the time between steps (a) and (b) comprises at least 90 minutes.

19. The method of claim 9, wherein the time between steps (a) and (b) comprises about 180 minutes.
